(19) United States
(12) Patent Application Publication
(10) Pub. No.: US 2023/0265424 A1
(43) Pub. Date: Aug. 24, 2023

(54) METHODS OF TREATING AND PREVENTING BACTERIAL INFECTIONS

(71) Applicant: 1E Therapeutics Ltd., Rehovot (IL)

(72) Inventors: IDO BACHELET, Rehovot (IL); Alexander Rosenberg, Rehovot (IL); Adva Levy-Zamir, Rehovot (IL); Roni Oshri, Rehovot (IL); Nataly Mirlas-Neisberg, Rehovot (IL); Michal Pearl, Rehovot (IL); Tamar Zehavi-Chkola, Rehovot (IL); Itzhak Zander, Rehovot (IL)

(73) Assignee: 1E Therapeutics Ltd., Rehovot (IL)

(21) Appl. No.: 17/958,931

(22) Filed: Oct. 3, 2022

Related U.S. Application Data
(63) Continuation-in-part of application No. PCT/IL2021/051331, filed on Nov. 9, 2021.
(60) Provisional application No. 63/111,118, filed on Nov. 9, 2020.

(51) Int. Cl. C12N 15/113; A61K 47/54; A61P 31/04

(52) U.S. Cl. CPC C12N 15/113; A61K 47/554; A61P 31/04; C12N 2310/127; C12N 2310/3515

(57) ABSTRACT

Provided herein are DNAzymes conjugated to an organic moiety and methods of facilitating entry of DNAzymes into bacteria, utilizing same. Also provided are methods of targeting bacterial target genes, methods of treating or inhibiting the progression of bacterial infections, and methods of increasing susceptibility of bacteria to an antibiotic, using the described DNAzymes, which are optionally capable of silencing at least one target gene of bacteria and/or rendering bacteria susceptible to antibiotic treatment.

### RELATED APPLICATIONS

This application is a Continuation-in-Part of PCT/IL2021/051331, filed on Nov. 9, 2021, which claims the benefit of priority of U.S. Provisional Patent Application No. 63/111,118, filed on Nov. 9, 2020, the contents of which are incorporated herein by reference in their entirety.

### SEQUENCE LISTING

The instant application contains a Sequence Listing which has been submitted electronically in XML file format and is hereby incorporated by reference in its entirety. Said XML copy, created on Apr. 27, 2023, is named P-616663-US_SL.xml and is 52,356 bytes in size.

### FIELD OF DISCLOSURE

The present disclosure relates in general to DNAzymes conjugated to organic moieties, namely cyclic organic compounds having multiple rings and alkyl compounds, and methods of facilitating entry of DNAzymes into bacteria. In some aspects, the present disclosure relates in general to methods of targeting bacterial target genes, methods of treating or inhibiting the progression of bacterial infections, and methods of increasing susceptibility of bacteria to an antibiotic, using the conjugated DNAzymes.

### BACKGROUND

### DNAzymes

DNA enzymes (DNAzymes) are synthetic, catalytically-active DNA molecules that are able to specifically cleave target mRNA without requiring the involvement of cellular mechanisms such as the RNA-Induced Silencing Complex (RISC). DNAzymes have not been reported in nature and are typically generated by in-vitro selection. Moreover, DNAzymes are diverse structurally and mechanistically, and exhibit diverse secondary structures, metal ion dependencies, and catalysis kinetics.

DNAzymes typically consist of a catalytic core flanked by two arms that recognize its RNA target through Watson Crick base pairing and cleave RNA in a specific phosphodiester linkage. DNAzymes are a powerful tool for specific gene therapy due to their high specificity and catalytic efficiency, among other reasons because they are easy to synthesize and modify.

The therapeutic potential of DNAzymes has been demonstrated in diverse settings, including in antibiotic-resistant bacterial infections. The studies published to date have generally utilized either plasmid transfection or physical disruption methods such as electroporation to enable penetration of the DNAzyme into the bacteria cell, which requires traversal of a peptidoglycan-containing cell wall. Such methods may not be practicable in in-vivo therapeutic methods.

Improved methods of enhancing penetration of DNAzymes across bacterial cell walls are needed in the art.

### Steroids

Steroids are compounds that contain an optionally substituted gonane structure (shown below). The below structure may be modified, for example by the presence of double bonds, ring heteroatoms, and/or functional groups, without affecting the classification of the compound as a steroid.

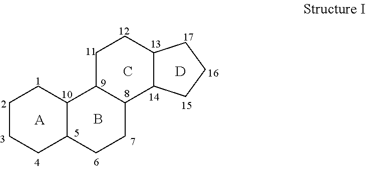

### Sterols

Sterols are a subset of steroids that contain at least 1 hydroxyl group. Certain sterols have the hydroxyl group in position 3 (shown in Structure III). Structure III may be modified, for example by the presence of double bonds, ring heteroatoms, and/or functional groups, without affecting the classification of the compound as a sterol.

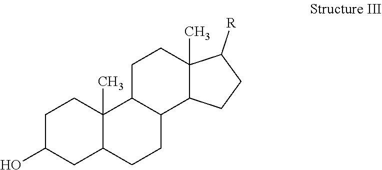

### Fatty Acids

Fatty acids are aliphatic chains, typically containing at least 6 carbon atoms, and having at least one carboxylic acid moiety. The chains can be either saturated or unsaturated, and either straight or branched. Structure XI is a general structure of a straight-chain, saturated fatty acid.

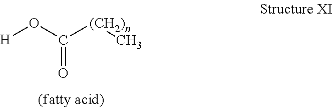

### SUMMARY

In certain aspects, provided herein are DNAzymes conjugated to an organic moiety and their use in treating a bacterial infection and potentiating antibiotic treatment in a subject in need thereof, and methods of facilitating entry of DNAzymes into bacteria.

In some aspects, provided herein is a DNAzyme conjugated to an organic moiety for use in treating a bacterial infection in a subject in need thereof. In some aspects, provided herein is a method of treating a bacterial infection in a subject in need thereof, comprising administering to the subject a DNAzyme conjugated to an organic moiety. In some aspects, provided herein is an article of manufacture comprising a DNAzyme conjugated to an organic moiety, being packaged in a packaging material and identified in print, in or on the packaging material for use in treating a bacterial infection. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

Where reference is made herein to a DNAzyme conjugated to an organic moiety, the organic moiety is selected from a) a cyclic organic compound having multiple rings; and b) an alkyl compound.

In some aspects, provided herein is a DNAzyme conjugated to an organic moiety for use in inhibiting progression of a bacterial infection in a subject in need thereof. In some aspects, provided herein is a method of inhibiting progression of a bacterial infection in a subject in need thereof, comprising administering to the subject a DNAzyme conjugated to an organic moiety. In some aspects, provided herein is an article of manufacture comprising a DNAzyme conjugated to an organic moiety, being packaged in a packaging material and identified in print, in or on the packaging material for use in inhibiting progression of a bacterial infection. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

In some aspects, provided herein is a DNAzyme conjugated to an organic moiety for use in increasing susceptibility of a bacterium to an antibiotic, in a subject having a bacterial infection. In some aspects, provided herein is a method of increasing susceptibility of a bacterium to an antibiotic in a subject in need thereof, comprising administering to the subject a DNAzyme conjugated to an organic moiety, In some aspects, provided herein is an article of manufacture comprising a DNAzyme conjugated to an organic moiety, being packaged in a packaging material and identified in print, in or on the packaging material for use in increasing susceptibility of a bacterium to an antibiotic. In some aspects, the DNAzyme targets a messenger RNA of an antibiotic resistance gene of the bacteria. In some aspects, the DNAzyme reduces expression of the protein product of an antibiotic resistance gene.

In some aspects, provided herein is a DNAzyme conjugated to an organic moiety for use in potentiating an antibiotic, in a subject having a bacterial infection. In some aspects, provided herein is a method of potentiating an antibiotic in a subject in need thereof, comprising administering to the subject a DNAzyme conjugated to an organic moiety. In some aspects, provided herein is an article of manufacture comprising a DNAzyme conjugated to an organic moiety, being packaged in a packaging material and identified in print, in or on the packaging material for use in potentiating an antibiotic. In some aspects, the DNAzyme targets a RNA that encodes an antibiotic resistance protein of the bacteria. In some aspects, the DNAzyme reduces expression of an antibiotic resistance protein.

In some aspects, provided herein is a DNAzyme conjugated to an organic moiety for use in reducing expression of a target bacterial RNA. In some aspects, provided herein is a method of reducing expression of a target bacterial RNA, comprising administering to the subject a DNAzyme conjugated to an organic moiety. In some aspects, provided herein is an article of manufacture comprising a DNAzyme, conjugated to an organic moiety, being packaged in a packaging material and identified in print, in or on the packaging material for use in reducing expression of a target bacterial RNA. In some aspects, the target RNA is essential to viability of to the bacteria. In some aspects, the target RNA is antibiotic resistance gene. In some embodiments, the target bacteria is disposed within a subject, who is, in some embodiments, a subject with a bacterial infection.

In some aspects, provided herein is a method of delivering a DNAzyme to the interior of a bacterial cell, comprising conjugating the DNAzyme to an organic moiety. In some aspects, the DNAzyme targets an RNA essential to viability of the bacteria. In some aspects, the DNAzyme reduces expression of the gene product of an RNA essential to viability of the bacteria. In some aspects, the DNAzyme targets an RNA product of an antibiotic resistance gene. In some aspects, the DNAzyme reduces expression of an antibiotic resistance protein.

In some aspects, provided herein is a method of delivering a DNAzyme across a bacterial cell wall, comprising conjugating the DNAzyme to an organic moiety. In some aspects, the DNAzyme targets an RNA essential to viability of the bacteria. In some aspects, the DNAzyme reduces expression of a protein essential to viability of the bacteria. In some aspects, the DNAzyme targets an RNA transcript of an antibiotic resistance gene. In some aspects, the DNAzyme reduces expression of an antibiotic resistance protein. In some embodiments, the cell wall comprises peptidoglycan.

In related aspects, the described DNAzymes target a RNA transcript. In further related aspects, the DNAzyme comprises, in 5′ to 3′ order: (i) a first substrate-binding domain (also referred to herein as the “5′ arm”) comprising a sequence that base pairs with a first region of the RNA transcript; (ii) a DNAzyme catalytic core and (iii) a second substrate-binding domain (also referred to herein as the “3′ arm”) comprising a sequence that base pairs with a second region of the RNA transcript positioned 5′ to the first region of the RNA transcript. In yet further related embodiments, upon binding of the DNAzyme to the RNA transcript, the DNAzyme catalytic core cleaves the RNA transcript. In certain aspects, the DNAzyme is capable of silencing at least one target gene of a bacterium.

In certain aspects, there is provided a pharmaceutical composition comprising the described conjugated DNAzymes, and a pharmaceutically acceptable carrier or diluent.

In certain aspects, there is provided an article of manufacture comprising the described conjugated DNAzymes.

In certain aspects, there is provided an article of manufacture comprising the described conjugated DNAzymes, and an antibiotic.

In certain aspects, there is provided a method of treating or preventing a bacterial infection in a subject in need thereof, the method comprising administering to the subject the described DNAzymes, thereby treating or preventing the bacterial infection in the subject.

In certain aspects, there is provided a method of treating or preventing a bacterial infection in a subject in need thereof, the method comprising administering to the subject the described DNAzymes, and an antibiotic, thereby treating or preventing the bacterial infection in the subject.

In certain aspects, there is provided a described DNAzyme, for use in treating or preventing a bacterial infection in a subject in need thereof.

In certain aspects, the DNAzyme is a 10-23 type DNAzyme molecule.

In certain aspects, the DNAzyme is an 8-17 type DNAzyme molecule.

In related aspects, the DNAzyme comprises modified nucleotides. In further related aspects, modified nucleotides comprise 2′-FANA. In further related aspects, the majority of the nucleotides comprise a 2′-FANA modification. In further related aspects, all of the nucleotides comprise a 2′-FANA modification.

In certain aspects, the bacterium is a Gram positive bacteria.

In certain aspects, the bacterium is a Gram negative bacteria.

In certain aspects, the bacterium is selected from the group consisting of a Enterococcus faecium, a Staphylococcus aureus, a Klebsiella pneumoniae, an Acinetobacter baumannii, a. Pseudomonas aeruginosa and an Enterobacter. In certain aspects, the bacterium is a Klebsiella pneumoniae.

In certain embodiments, the described conjugated DNAzyme is administered to a subject in need together with an antibiotic. In certain aspects, the DNAzyme and the antibiotic are in a co-formulation. In other aspects, the DNAzyme and the antibiotic are in separate formulations.

In certain aspects, the subject is a human subject. In other aspects, the subject is a non-human subject.

In certain aspects, the antibiotic is a β-lactam. In other aspects, the antibiotic is a carbapenem. In other aspects, the antibiotic is a penicillin. In other aspects, the antibiotic is a cephalosporin. In other aspects, the antibiotic is a monobactam. In other aspects, the antibiotic is selected from the group consisting of penicillin, methicillin, oxacillin, cephalosporin, aztreonam, cefoxitin, carbapenem, imipenem and meropenein.

In certain aspects, the described DNAzyme comprises or consists of ribonucleotides, deoxyribonucleotides, or a combination thereof.

In certain aspects, the oligonucleotide (DNAzyme) is formulated with or attached to a permeability enhancing moiety. In certain aspects, the permeability enhancing moiety is a cholesterol moiety.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The subject matter regarded as the deoxyribozymes (DNAzymes) disclosed herein is particularly pointed out and distinctly claimed in the concluding portion of the specification. The DNAzymes, however, both as to organization and method of operation, together with objects, features, and advantages thereof, may best be understood by reference to the following detailed description when read with the accompanying drawings in which:

FIG. 1A illustrates a schematic illustration of the binding of 10-23 DNAzyme to its target RNA. FIG. 1B illustrates the conservation of the target region of the DNAzyme KPC-337 targeting the bla carbapenemase in clinical isolates of Klebsiella pneumoniae (ec number 3.5.2.6, uniport ID Q9F663).

FIG. 2A is a graph demonstrating entry of conjugated DNAzymes, wherein a DNAzyme was conjugated to cholesterol-TEG at the 3′ end, into both S. Aureus and P. aeruginosa bacteria. Depicted is mean fluorescent intensity (MFI) of the bacterial population after incubation with the fluorescent DNAzyme, as detected by flow cytometry (vertical axis) as a function of time (hours; horizontal axis) FIG. 2B is a graph demonstrating efficacy of DNAzymes in the presence of sub-toxic meropenem levels against various antibiotic resistance genes in Klebsiella pneumoniae. Optical density (OD₆₀₀) representative of bacterial density, is plotted (vertical axis) vs. time (hours; horizontal axis). FIG. 2C is a graph depicting the MIC (minimal inhibitory concentration) of meropenem alone, showing lack of toxicity even at 128 μg/ml.

FIGS. 3A-B demonstrate uptake of conjugated DNAzymes into Klebsiella pneumoniae, in media containing sub-toxic concentrations of meropenem. FIG. 3A is a chart depicting fluorescence (arbitrary units; vertical axis) as detected by flow cytometry, vs. time (hours; horizontal axis). FIG. 3B is a histogram depicting frequency (vertical axis) vs. MFI (horizontal axis) of the bacterial population after 4 hours of incubation with the fluorescent conjugated DNAzymes. Black, dark grey, and light gray lines depict basal fluorescence levels of the bacteria, bacteria incubated with unmodified DNAzymes, and bacteria incubated with DNAzymes with a cholesterol-TEG modification at the 3′ end, respectively.

FIGS. 4A-B are plots demonstrating bioactivity of conjugated KPC-337. FIG. 4A shows expression fold-change reduction of bla-KPC transcript levels (vertical axis) after addition of KPC-337 to Klebsiella pneumoniae bacterial culture. FIG. 4B shows beta-lactamase activity (vertical axis) as detected by a colorimetric assay. Experimental groups in FIGS. 4A-B are shown on horizontal axis.

FIGS. 5A-C illustrate antibiotic sensitization of a resistant Klebsiella pneumoniae strain by conjugated DNAzyme (KPC-337) treatment. Top panel of FIG. 5A shows OD₆₀₀, illustrated colorimetrically, indicating relative bacterial growth inhibition at the indicated meropenem concentrations. Middle column of bottom panel of FIG. 5A shows reduction in MIC (minimal inhibitory concentration) in the same experiment. FIG. 5B is a picture showing lack of colony formation after plating, indicating a bactericidal effect of KPC-337. Right column of bottom panel of FIG. 5A shows reduction in MBC (minimal bactericidal concentration) in the same experiment. FIG. 5C is a plot showing reduction of colony forming units per milliliter (CFU/ml; vertical axis) in bacteria culture vs. time (horizontal axis) by KPC-337 in the presence of a sub-toxic meropenem concentration. All experiments in this figure include a control of a random, non-catalytic sequence designated scramble (“SCR”).

FIGS. 6A-B are plots illustrating a bactericidal effect of conjugated KPC-337 in the presence of a sub-toxic meropenem concentration on an antibiotic resistant Klebsiella pneumoniae strain (ATCC® BAA-1705™) in lung tissue, as measured by colony forming units per milliliter (CFU/ml; vertical axis). Experimental groups in FIGS. 4A-B are shown on horizontal axis. SCR indicates a control (random, non-catalytic) sequence. Treatment in meropenem alone was not effective. FIG. 6A depicts cells infecting the tissue, and FIG. 6B depicts free living cells that remained in the growth media.

FIGS. 7A-B are plots illustrating the beneficial effect of conjugated KPC-337 in the presence of a sub-toxic meropenem concentration in in vivo models. FIG. 7A shows a reduction of toxicity of ATCC® BAA-1705™ infection, as expressed by moth larvae viability (vertical axis) vs study day (horizontal axis). SCR indicates a control (random, non-catalytic) sequence. FIG. 7B shows reduction bacterial cell count of ATCC® BAA-1705™ infecting a murine thigh, as expressed by CFU (vertical axis). Experimental groups are shown on horizontal axis. NT and MP indicate untreated and antibiotic treatment alone.

FIG. 8 is a plot illustrating lack of toxicity of conjugated KPC-337 to human HT-29 cells at bioactive concentrations (e.g. <5 μM). Percent toxicity (vertical axis) is plotted against DNAzyme concentration (horizontal axis). SCR is a control (random, non-catalytic) sequence.

FIG. 9 is a plot illustrating the sensitization of methicillin-resistant Staphylococcus aureus (MRSA) to (a sub-toxic concentration of) cefoxitin by conjugated DNAzymes targeting resistance genes. Bacterial growth is expressed as colony forming units per milliliter (CFU/ml; vertical axis) in bacteria culture as vs. time (horizontal axis). Black and gray circles (“SA” and “MRSA”) indicate untreated bacteria and bacteria treated with antibiotic alone. Other symbols indicate DNAzyme in combination with the indicated DNAzymes.

FIG. 10 is a plot illustrating a dose response assay for a conjugated DNAzyme, mecA-658 (SEQ ID NO: 20). targeting the resistant gene mecA in MRSA. Optical density (OD₆₀₀) representative of bacterial density, is plotted (vertical axis) vs. time (hours; horizontal axis). Black and gray circles (“SA” and “MRSA”) indicate untreated bacteria and bacteria treated with antibiotic alone. Other symbols indicate the indicated concentrations of the DNAzyme.

FIG. 11 is a plot illustrating that conjugated DNAzymes with various arm lengths are against MRSA. Black and gray circles (“SA” and “MRSA”) indicate untreated bacteria and bacteria treated with antibiotic alone. Other symbols indicate DNAzyme in combination with the indicated DNAzymes.

FIG. 12 is a plot illustrating sustained (≥44-hour) MRSA growth inhibition by a single dose of conjugated DNAzyme, FemA-545 (SEQ ID NO: 25), in the presence of sub-optimal antibiotic concentration. Black and gray circles (“SA” and “MRSA”) indicate untreated bacteria and bacteria treated with antibiotic alone, and gray squares indicate DNAzyme-treated bacteria.

### DETAILED DESCRIPTION

In the following detailed description, numerous specific details are set forth in order to provide a thorough understanding of the described DNAzymes. However, it will be understood by those skilled in the art that the conjugated DNAzymes and uses thereof may be practiced without these specific details. In other instances, well-known methods, procedures, and components have not been described in detail so as not to obscure the DNAzymes presented herein.

The present disclosure, in some embodiments thereof, relates to DNAzymes conjugated to an organic moiety, their use in treating a bacterial infection and potentiating antibiotic treatment in a subject in need thereof, and methods of facilitating entry of DNAzymes into bacteria. Where reference is made herein to a DNAzyme conjugated to an organic moiety, the organic moiety is selected from a) a cyclic organic compound having multiple rings; and b) an alkyl compound.

In some embodiments, provided herein is a DNAzyme conjugated to an organic moiety for use in treating a bacterial infection in a subject in need thereof. In some embodiments, provided herein is a method of treating a bacterial infection in a subject in need thereof, comprising administering to the subject a DNAzyme conjugated to an organic moiety. In some embodiments, provided herein is an article of manufacture comprising a DNAzyme conjugated to an organic moiety, being packaged in a packaging material and identified in print, in or on the packaging material for use in treating a bacterial infection. In some embodiments, the DNAzyme is capable of silencing a target gene of a bacterium. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein.

In some embodiments, provided herein is a DNAzyme conjugated to an organic moiety for use in inhibiting progression of a bacterial infection in a subject in need thereof. In some embodiments, provided herein is a method of inhibiting progression of a bacterial infection in a subject in need thereof, comprising administering to the subject a DNAzyme conjugated to an organic moiety. In some embodiments, provided herein is an article of manufacture comprising a DNAzyme conjugated to an organic moiety, being packaged in a packaging material and identified in print, in or on the packaging material for use in inhibiting progression of a bacterial infection. In some embodiments, the DNAzyme is capable of silencing a target gene of a bacterium. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein.

In some embodiments, provided herein is a DNAzyme conjugated to an organic moiety for use in increasing susceptibility of a bacterium to an antibiotic, in a subject having a bacterial infection. In some embodiments, provided herein is a method of increasing susceptibility of a bacterium to an antibiotic in a subject in need thereof, comprising administering to the subject a DNAzyme conjugated to an organic moiety. In some embodiments, provided herein is an article of manufacture comprising a DNAzyme conjugated to an organic moiety, being packaged in a packaging material and identified in print, in or on the packaging material for use in increasing susceptibility of a bacterium to an antibiotic. In some embodiments, the DNAzyme is capable of silencing a target gene of a bacterium. In some embodiments, the DNAzyme targets a messenger RNA of an antibiotic resistance gene of the bacteria. In some embodiments, the DNAzyme reduces expression of the protein product of an antibiotic resistance gene.

In some embodiments, provided herein is a DNAzyme conjugated to an organic moiety for use in potentiating an antibiotic, in a subject having a bacterial infection. In some embodiments, provided herein is a method of potentiating an antibiotic in a subject in need thereof, comprising administering to the subject a DNAzyme conjugated to an organic moiety. In some embodiments, provided herein is an article of manufacture comprising a DNAzyme conjugated to an organic moiety, being packaged in a packaging material and identified in print, in or on the packaging material for use in potentiating an antibiotic. In some embodiments, the DNAzyme is capable of silencing a target gene of a bacterium. In some embodiments, the DNAzyme targets a RNA that encodes an antibiotic resistance protein of the bacteria. In some embodiments, the DNAzyme reduces expression of an antibiotic resistance protein.

In some embodiments, provided herein is a DNAzyme conjugated to an organic moiety for use in reducing expression of a target bacterial RNA, in a subject having a bacterial infection. In some embodiments, provided herein is a method of reducing expression of a target bacterial RNA in a subject in need thereof, comprising administering to the subject a DNAzyme conjugated to an organic moiety. In some embodiments, provided herein is an article of manufacture comprising a DNAzyme conjugated to an organic moiety, being packaged in a packaging material and identified in print, in or on the packaging material for use in reducing expression of a target bacterial RNA. In some embodiments, the DNAzyme is capable of silencing a target gene of a bacterium. In some embodiments, the target RNA is essential to viability of the bacteria. In some embodiments, the target RNA is antibiotic resistance gene.

In some embodiments, reducing expression comprises reducing a concentration of a target RNA. In other embodiments, reducing expression comprises reducing a concentration of a protein product of a target RNA. In some embodiments, reducing expression denotes reducing a concentration of a target RNA. In other embodiments, reducing expression denotes reducing a concentration of a protein product of a target RNA.

In some embodiments, provided herein is a method of delivering a DNAzyme to the interior of a bacterial cell, comprising conjugating the DNAzyme to an organic moiety. In some embodiments, the DNAzyme targets an RNA essential to viability of the bacteria. In some embodiments, the DNAzyme reduces expression of the gene product of an RNA essential to viability of the bacteria. In some embodiments, the DNAzyme is capable of silencing a target gene of a bacterium. In some embodiments, the DNAzyme targets an RNA product of an antibiotic resistance gene. In some embodiments, the DNAzyme, reduces expression of an antibiotic resistance protein.

In some embodiments, provided herein is a method of delivering a DNAzyme across a bacterial cell wall, comprising conjugating the DNAzyme to an organic moiety and contacting a bacterial cell with the conjugated DNAzyme. In some embodiments, the DNAzyme targets an RNA essential to viability of the bacteria. In some embodiments, the DNAzyme reduces expression of the gene product of an RNA essential to viability of the bacteria. In some embodiments, the DNAzyme targets an RNA product of an antibiotic resistance gene. In some embodiments, the DNAzyme reduces expression of an antibiotic resistance protein.

In some embodiments, provided herein are use of the described conjugated DNAzymes for targeting bacterial genes.

As provided herein, the described moieties enable DNA enzymes (DNAzymes) to penetrate bacteria cell walls and subsequently target and cleave RNA transcripts of selected bacterial genes. In some embodiments, administering these DNAzymes to bacteria is cytotoxic to the bacteria. In some embodiments, administering these DNAzymes to bacteria renders the bacteria susceptible to antibiotics.

### Organic Moieties

In some embodiments, the described organic moiety is an organic compound. In some embodiments, the organic moiety is aromatic. In some embodiments, the organic moiety is non-aromatic. In some embodiments, the organic moiety is aliphatic.

In some embodiments, the organic moiety is directly conjugated to the DNAzyme. In other embodiments, the organic moiety is conjugated to the DNAzyme via a linker.

In some embodiments, the described organic moiety is a cyclic organic compound, wherein the cyclic organic compound comprises multiple rings. In some embodiments, 2-6 rings are present in the compound. In some embodiments, 2-5 rings are present. In some embodiments, 2-4 rings are present. In some embodiments, 3 rings are present. In some embodiments, 4 rings are present.

In some embodiments, the cyclic organic compound is directly conjugated to the DNAzyme, in other embodiments, the cyclic organic compound is conjugated to the DNAzyme via a linker.

In some embodiments, the described organic moiety is a steroid, used herein to refer to compounds that contain an optionally substituted gonane structure (I). In some embodiments, the ring systems of the gonane structure are modified with up to 3 double bonds. In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, the gonane structure is substituted with up to 3 additional functional groups at one or more of positions 1-17. In some embodiments, the functional groups are selected from OH, NO₂, NH₃, F, Cl, Br, I, NR₃, C(O)NR₃, unsubstituted alkyl, substituted alkyl, unsubstituted alkenyl, substituted alkenyl, unsubstituted alkynyl, substituted alkynyl, unsubstituted alkoxy, or substituted alkoxy; wherein R is, independently in each instance, H, unsubstituted alkyl, or substituted alkyl; each of which represents a separate embodiment. In some embodiments, substituted alkyl, substituted alkenyl, or substituted alkynyl contains 1-5 substitutions independently selected from OH, NO₂, NH₃, F, Cl, Br, I, NR₃, C(O)NR₃, where R is defined as above; each of which represents a separate embodiment. The above modifications may be freely combined, and each combination represents a separate embodiment.

In some embodiments, the steroid is directly conjugated to the DNAzyme. In other embodiments, the steroid is conjugated to the DNAzyme via a linker.

In some embodiments, the junction of the A and B rings has a trans configuration. In other embodiments, the junction of the A and B rings has a cis configuration.

In some embodiments, the steroid is physiologically acceptable.

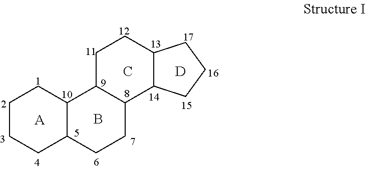

In some embodiments, the described organic moiety has the following structure II:

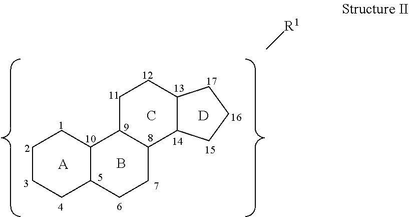

where n=1-3; and

R¹ is, independently in each instance, OH, NO₂, NH₃, F, Cl, Br, I, N(R²)₃, C(O)N N(R²)₃, unsubstituted alkyl, substituted alkyl, unsubstituted alkenyl, substituted alkenyl, unsubstituted alkynyl, substituted alkynyl, unsubstituted alkoxy, substituted alkoxy; wherein R² is, independently in each instance, H, unsubstituted alkyl, or substituted alkyl; each of which represents a separate embodiment. In some embodiments, substituted alkyl, substituted alkenyl, or substituted alkynyl contains 1-5 substitutions independently selected from OH, NO₂, NH₃, F, Cl, Br, I, N N(R²)₃, C(O)N N(R²)₃, where R² is defined as above; each of which represents a separate embodiment.

As used herein, “alkyl” refers to a monovalent saturated hydrocarbon moiety. In some embodiments, an alkyl moiety is linear. In some embodiments, an alkyl moiety is branched. In some embodiments, an alkyl moiety has between 1-20 carbon atoms. In some embodiments, an alkyl moiety has between 1-12 carbon atoms. In some embodiments, an alkyl moiety has between 1-8 carbon atoms. In some embodiments, an alkyl moiety has between 1-6 carbon atoms. In some embodiments, an alkyl moiety has between 1-4 carbon atoms. In some embodiments, an alkyl moiety has between 1-3 carbon atoms. Examples of alkyl. moieties include, but are not limited to methyl, ethyl, npropyl, i-propyl, n-butyl, s-butyl, t-butyl, i-butyl, a pentyl moiety, a hexyl moiety, cyclopropyl, cyclobutyl, cyclopentyl, and cyclohexyl. Each of the aforementioned possibilities represents a separate embodiment.

As used herein, “alkenyl” refers to a monovalent hydrocarbon moiety containing at least two carbon atoms and one or more nonaromatic carbon-carbon double bonds. In some embodiments, an alkenyl moiety is linear. In some embodiments, an alkenyl moiety is branched. In some embodiments, an alkenyl moiety has between 1-12 carbon atoms. In some embodiments, an alkenyl moiety has between 1-8 carbon atoms. In some embodiments, an alkenyl moiety has between 1-6 carbon atoms. In some embodiments, an alkenyl moiety has between 1-4 carbon atoms. In some embodiments, an alkenyl moiety has between 1-3 carbon atoms. Examples of alkenyl moieties include, but are not limited to vinyl, propenyl, butenyl, and cyclohexenyl. Each of the aforementioned possibilities represents a separate embodiment.

As used herein, “alkynyl” refers to a monovalent hydrocarbon radical moiety containing at least two carbon atoms and one or more carbon-carbon triple bonds. In some embodiments, an alkynyl moiety is branched. In some embodiments, an alkynyl moiety has between 1-20 carbon atoms. In some embodiments, an alkynyl moiety has between 1-12 carbon atoms. In some embodiments, an alkynyl moiety has between 1-8 carbon atoms. In some embodiments, an alkynyl moiety has between 1-6 carbon atoms. In some embodiments, an alkynyl moiety has between 1-4 carbon atoms. In some embodiments, an alkynyl moiety has between 1-3 carbon atoms. Examples of alkynyl moieties include, but are not limited to ethynyl, propynyl, and butynyl. Each of the aforementioned possibilities represents a separate embodiment.

As used herein, “alkoxy” refers to a monovalent and saturated hydrocarbon moiety, wherein the hydrocarbon includes a single bond to an oxygen atom, e.g., CH₃CH₂—O· for ethoxy. Alkoxy substituents bond to the compound which they substitute through this oxygen atom of the alkoxy substituent. In some embodiments, an alkoxy moiety is linear. In some embodiments, are alkoxy moiety is branched. In some embodiments, an alkoxy moiety has between 1-20 carbon atoms. In some embodiments, an alkoxy moiety has between 1-12 carbon atoms. In some embodiments, an alkoxy moiety has between 1-8 carbon atoms. In some embodiments, an alkoxy moiety has between 1-6 carbon atoms. In some embodiments, an alkoxy moiety has between 1-4 carbon atoms. In some embodiments, an alkyl moiety has between 1-3 carbon atoms. Examples of alkoxy moieties include, but are not limited to methoxy, ethoxy, n-propoxy, i-propoxy, n-butoxy, s-butoxy, t-butoxy, i-butoxy, a pentoxy moiety, a hexoxy moiety, cyclopropoxy, cyclobutoxy, cyclopentoxy, and cyclohexoxy. Each of the aforementioned possibilities represents a separate embodiment.

In some embodiments, the steroid is a gonane alcohol. In some embodiments, the gonane alcohol is physiologically acceptable. In some embodiments, a sterol is a molecule derived from gonane by replacement of a hydrogen atom at any of positions 1-17 by a hydroxyl group.

In some embodiments, the steroid is a sterol (Structure III). In some embodiments, the steroid differs from gonane by replacement of a hydrogen atom in position 3 by a hydroxyl group. In some embodiments, the steroid differs from gonane by replacement of a hydrogen atom in position 10 by a methyl group. In some embodiments, the steroid differs from gonane by replacement of a hydrogen atom in position 13 by a methyl group. In some embodiments, the steroid differs from gonane by replacement of a hydrogen atom in position 17 by an alkyl group. In some embodiments, the steroid differs from gonane by (a) replacement of a hydrogen atom in position 3 by a hydroxyl group; (b) replacement of a hydrogen atom in position 10 by a methyl group; (c) replacement of a hydrogen atom in position 13 by a methyl group; and (d) replacement of a hydrogen atom in position 17 by an alkyl group, each of which, alone or in combination, represents a separate embodiment. In some embodiments, the steroid has at least 2 of aforementioned elements (a)-(d). In some embodiments, the steroid has at least 3 of aforementioned elements (a)-(d). In some embodiments, the steroid has all of aforementioned elements (a)-(d). The above modifications may be freely combined, and each combination represents a separate embodiment.

In some embodiments, the junction of the A and B rings has a trans configuration. In other embodiments, the junction of the A and B rings has a cis configuration.

In some embodiments, the sterol is directly conjugated to the DNAzyme. In other embodiments, the sterol is conjugated to the DNAzyme via a linker.

In some embodiments, the sterol is physiologically acceptable.

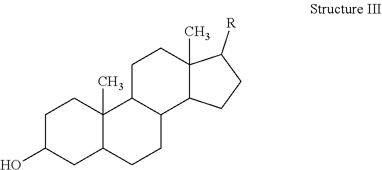

In some embodiments, a sterol is defined as per CHEBI: 15889.

In some embodiments, the described organic moiety has the following Structure IV:

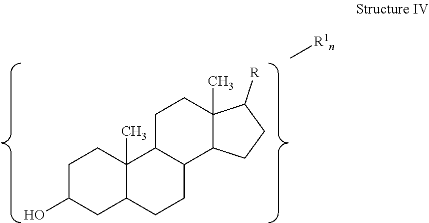

where n=1-3; and

where R¹ is defined as set forth hereinabove.

In some embodiments, the steroid is cholesterol (Structure V; PubChem CID 5997). cholestan-3-ol (Structure VI; PubChem CID 10992748), or a derivative of cholesterol or cholestan-3-ol; each of which represents a separate embodiment. In some embodiments, the sterol comprises a 3beta-hydroxy group. In some embodiments, the sterol has a double bond at the 5,6-position. In some embodiments, the sterol comprises an alkyl group at position 17. In some embodiments, the sterol comprises a 3beta-hydroxy group; a double bond at the 5,6-position; and an alkyl group at position 17. In some embodiments, the alkyl group contains 5-8 carbon atoms. Cholesterol is a non-limiting example of a cholestane having a 3beta-hydroxy group; a double bond at the 5,6-position; and an alkyl group at position 17.

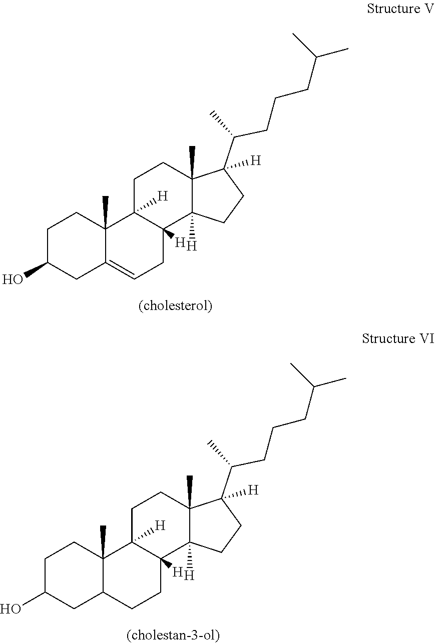

In some embodiments, the cholesterol, cholestan-3-ol, or derivative thereof is directly conjugated to the DNAzyme. In other embodiments, the cholesterol, cholestan-3-ol, or derivative to thereof is conjugated to the DNAzyme via a linker.

In some embodiments, the ring systems of the derivative of cholesterol or cholestan-3-ol are modified with up to 3 double bonds (3 additional double bonds, in the case of a cholesterol derivative). In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, as depicted in Structures VII and VIII, the cholesterol or cholestan-3-ol structure is substituted with up to 3 additional functional groups at one or more positions, which may be any of the functional groups described herein, each of which represents a separate embodiment. In Structures VII and VIII, n and R¹ are defined as set forth hereinabove.

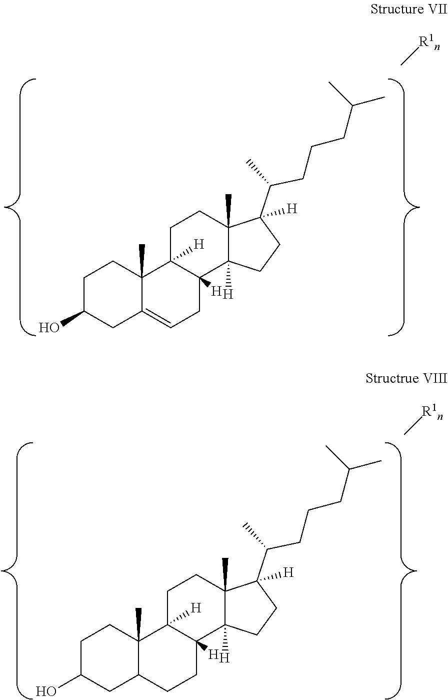

In some embodiments, the steroid is cholestane (Structure IX; PubChem CID 637620) or a derivative of cholestane. In some embodiments, the ring systems of the derivative of cholestane are modified with up to 3 double bonds. In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, the cholestane structure is substituted with up to 3 additional functional groups at one or more of positions 1-17, which may be any of the functional groups described herein, each of which represents a separate embodiment.

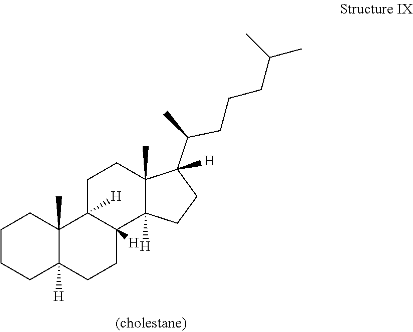

In some embodiments, the cholestane or derivative thereof is directly conjugated to the DNAzyme. In other embodiments, the cholestane or derivative thereof is conjugated to the DNAzyme via a linker.

In some embodiments, the ring systems of the derivative of cholestane are modified with up to 3 double bonds. In some embodiments, the ring systems of the gonane structure are modified with up to 3 heteroatoms. In some embodiments, the heteroatoms are selected from nitrogen, oxygen, and sulfur; each of which represents a separate embodiment. In some embodiments, as depicted in Structure X, the cholestane structure is substituted with up to 3 additional functional groups at one or more positions, which may be any of the functional groups described herein, each of which represents a separate embodiment. In the below Structure X, n and R¹ are defined as set forth hereinabove.

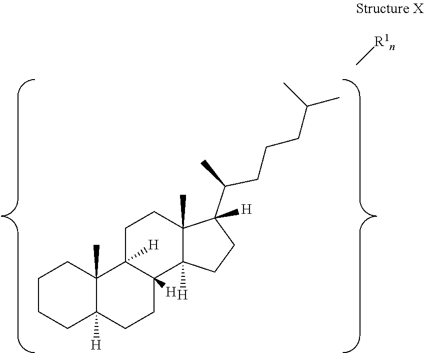

In some embodiments, the described sterol is selected from the group consisting of cholesterol, β-sitosterol, campesterol, stigmasterol, and ergosterol; and derivatives of any of aforementioned compounds with 2 or fewer substitutions.

In some embodiments, the organic moiety is a bile acid or bile alcohol. In some embodiments, the terms bile acids and bile alcohols refer to steroids with a core structure of seventeen carbon atoms arranged in four fused rings, i.e., three cyclohexane rings (rings A-C) and one cyclopentane ring (ring D), together with a five or eight carbon sidechain terminating in a carboxylic acid group (or hydroxyl in the bile alcohols). In related embodiments, bile acids and bile alcohols further contain hydroxyl groups at positions C3, C7 and C12, and methyl groups at in positions C18 and C19. In related embodiments, the junction of the A and B rings of bile acids has a cis or chair configuration. In certain embodiments, the bile acid or bile alcohol is selected from a C₂₇ bile alcohols, a C₂₇ bile acid, and a C₂₄ bile acid.

In some embodiments, the bile alcohol is a C₂₇ bile alcohol. In some embodiments, the C₂₇ bile alcohol is physiologically acceptable.

In some embodiments, the bile acid is a C₂₇ bile acid. In some embodiments, the C₂₇ bile acid is physiologically acceptable.

In some embodiments, the bile acid is a C₂₄ bile acid. in some embodiments, the C₂₄ bile acid is physiologically acceptable.

In some embodiments, the described sterol is a phytosterol, non-limiting examples of which include β-sitosterol, campesterol, stigmasterol, stigmastanol, campestanol, and brassicasterol. In some embodiments, the phytosterol is physiologically acceptable.

In some embodiments, the described steroid is a physiologically acceptable steroid selected from the group consisting of androstane, cholestane, gorgostane, bufanolide, ergostane, poriferastane, campestane, estrane, pregnane, cardanolide, furostan, spirostan, cholane, gonane, and stigmastane.

In some embodiments, the organic moiety is an alkyl moiety. In some embodiments, the alkyl moiety is aliphatic. In some embodiments, the alkyl moiety is directly conjugated to the DNAzyme. In other embodiments, the alkyl moiety is conjugated to the DNAzyme via a linker.

In some embodiments, the alkyl moiety comprises a chain of 8-20 carbons, meaning that that total number of carbon atoms in the chain are 8-20. In some embodiments, the alkyl moiety is straight chained. In other embodiments, the alkyl moiety is branched. In some embodiments, the alkyl moiety is unsubstituted. In other embodiments, the alkyl moiety is substituted. In some embodiments, the carbons of the alkyl moiety are replaced with up to 3 heteroatoms. In some embodiments, the heteroatoms are selected from nitrogen, oxygen, and sulfur; each of which represents a separate embodiment. In some embodiments, the chain is substituted with up to 3 functional groups at one or more positions, which may be any of the functional groups described herein, each of which represents a separate embodiment.

In some embodiments, the alkyl moiety is a fatty acid. In some embodiments, the alkyl moiety of the fatty acid is aliphatic. In some embodiments, the fatty acid has Structure XI.

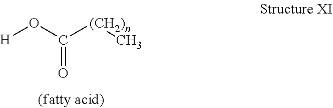

In some embodiments, the fatty acid moiety contains at least 9 carbons. In some embodiments, the fatty acid is selected from the group consisting of: nonanoyl (C₉); capryl (C₁₀); undecanoyl (C₁₁); lauroyl (C₁₂); tridecanoyl (C₁₃); myristoyl (C₁₄); pentadecanoyl (C₁₅), palmitoyl (C₁₆); phytanoyl (methyl substituted C₁₆); heptadecanoyl (C₁₇); stearoyl (C₁₈); nonadecanoyl (C₁₉); arachidoyl (C₂₀); heniecosanoyl (C₂₁); behenoyl (C₂₂); trucisanoyl (C₂₃); or lignoceroyl (C₂₄).

In some embodiments, the fatty acid moiety comprises a chain of 8-20 carbons. In some embodiments, the fatty acid moiety is straight chained. In other embodiments, the fatty acid moiety is branched. In some embodiments, the fatty acid moiety is unsubstituted. In other embodiments, the fatty acid moiety is substituted. In some embodiments, the carbons of the fatty acid moiety are replaced with up to 3 heteroatoms. In some embodiments, the heteroatoms are selected from nitrogen, oxygen, and sulfur; each of which represents a separate embodiment. In some embodiments, the chain is substituted with up to 3 functional groups at one or more positions, which may be any of the functional groups described herein, each of which represents a separate embodiment.

In some embodiments, the fatty acid moiety is directly conjugated to the DNAzyme. In other embodiments, the fatty acid moiety is conjugated to the DNAzyme via a linker.

In some embodiments, the organic moiety is a fatty acid amide. In some embodiments, a fatty acid amide has the structure set forth in Structure XII, wherein R represents an alkyl group. In some embodiments, R is a chain of 8-20 carbons. In some embodiments, R is straight chained. In other embodiments, R is branched. In some embodiments, R is unsubstituted. In other embodiments, R is substituted.

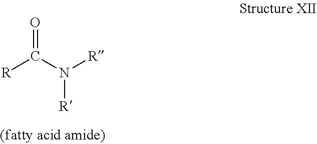

In some embodiments, R′ is H. In some embodiments, R″ is the described DNAzyme, indicating that the fatty acid amide is directly conjugated to the DNAzyme. In other embodiments, R″ is a linker, which is covalently bound to the DNAzyme, indicating that the fatty acid moiety is conjugated to the DNAzyme via a linker.

In some embodiments, the DNAzyme molecule is formulated with or attached to a permeability enhancing moiety.

Permeability enhancing moieties include, without being limited to, lipidic moieties (i.e. naturally occurring or synthetically produced lipids) such as a cholesterol moiety, cholic acid, a thioether, e.g., beryl-S-tritylthiol, a thiocholesterol, an aliphatic chain, dodecandiol or undecyl residues, a phospholipid, e.g.. di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, an octadecyl amine or hexylamino-carbonyloxycholesterol moiety, a steroid, a sphingosine, a ceramide, or a fatty acid moiety. The fatty acid moiety can be, e.g., any fatty acid which contains at least eight carbons. For example, the fatty acid can be, e.g., a nonanoyl (C₉); capryl (C₁₀); undecanoyl (C₁₁); lauroyl (C₁₂); tridecanoyl (C₁₃); myristoyl (C₁₄), pentadecanoyl (C₁₅); palmitoyl (C₁₆); phytanoyl (methyl substituted C₁₆); heptadecanoyl (C₁₇); stearoyl (C₁₈); nonadecanoyl (C₁₉); arachidoyl (C₂₀); heniecosanoyl (C₂₁); behenoyl (C₂₂); trucisanoyl (C₂₃), or a lignoceroyl (C₂₄) moiety. The cell-penetrating moiety can also include multimers (e.g., a composition containing more than one unit) of octyl-glycine, 2-cyclohexylalanine, or benzolylphenylalanine. The cell-penetrating moiety contains an unsubstituted or a halogen-substituted (e.g., chloro) biphenyl moiety. Substituted biphenyls are associated with reduced accumulation in body tissues, as compared to compounds with a non-substituted biphenyl. Reduced accumulation in bodily tissues following administration to a subject is associated with decreased adverse side effects in the subject.

According to one embodiment, the permeability enhancing moiety is a polysaccharide (e.g. mannose), a synthetic nucleoside base, an inverted nucleoside base, a cholesterol, other sterols (e.g. methyl sterols, dimethyl sterols), a lipid, a membrane lipid (e.g. phospholipids, glycolipids), and a synthetic lipid.

According to a specific embodiment, the nucleic acid oligonucleotide is conjugated to a permeability enhancing moiety is cholesterol. According to one embodiment, the cholesterol is linked directly or via a linker to the 5′ or 3′ terminus (or both) of the oligonucleotide.

### Linkers

As mentioned, in some embodiments, organic moieties are conjugated to DNAzymes via a linker. In some embodiments, the linker is an organic compound. In some embodiments, the organic compound is aromatic. In some embodiments, the organic compound is non-aromatic. In some embodiments, the organic compound is aliphatic.

In some embodiments, the linker is an alkyl moiety. In some embodiments, the alkyl moiety contains at least 9 carbons. In some embodiments, the alkyl is selected from the group consisting of: nonane (C₉), decane (C₁₀); undecane (C₁₁); dodecane (C₁₂), tridecane (C₁₃), tetradecane (C₁₄), pentadecane (C₁₅); hexadecane (C₁₆); heptadecane (C₁₇); octadecane (C₁₈); nonadecane (C₁₉); icosane (C₂₀); heniecosane (C₂₁); docosane (C₂₂); tricosane (C₂₃); or tetracosane (C₂₄).

In some embodiments, the linker comprises a chain of 8-20 carbons. In some embodiments, the linker is straight chained. In other embodiments, the carbon chain is branched. In some embodiments, the carbon chain is unsubstituted. In other embodiments, the carbon chain is substituted. In some embodiments, the carbons of the chain are replaced with up to 3 heteroatoms. In some embodiments, the heteroatoms are selected from nitrogen, oxygen, and sulfur; each of which represents a separate embodiment. In some embodiments, the chain is substituted with up to 3 functional groups at one or more positions, which may be any of the functional groups described herein, each of which represents a separate embodiment.

In some embodiments, the linker is a glycol. In some embodiments, the linker is a glycol polymer. In some embodiments, the linker is an ethylene glycol polymer. In some embodiments, the glycol or glycol polymer comprises a chain of 8-20 carbons. In some embodiments, the linker is straight chained. In other embodiments, the carbon chain is branched. In some embodiments, the linker is triethylene glycol, shown conjugated to cholesterol, to form a structure referred to as cholesterol-TEG (or cholTEG), in Structure XIII.

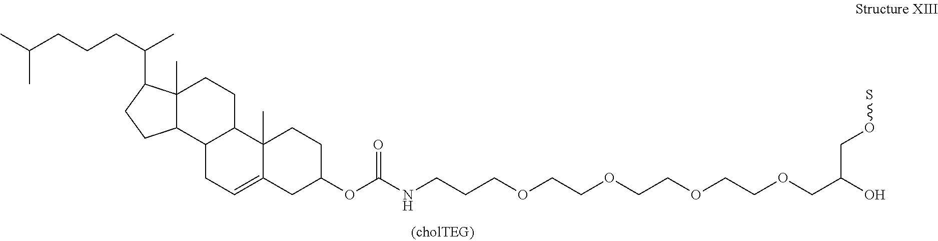 [SMILES: CC(C)CCCC(C)C1CCC2C3CC=C4CC(OC(=O)NCCCOCCOCCOCCOCC(O)COS)CCC4(C)C3CCC12C]

It is recognized that the organic moiety and linker can both be an alkyl moiety. Solely for exemplification. Structure XIV illustrates a hexadecanamide moiety (which falls within the definition herein of an alkyl moiety) conjugated via a branched hexanol linker (which falls within the definition herein of an alkyl moiety) to a DNAzyme. The depicted molecule is available commercially as Gene Link™ cat. no. 26-6621.

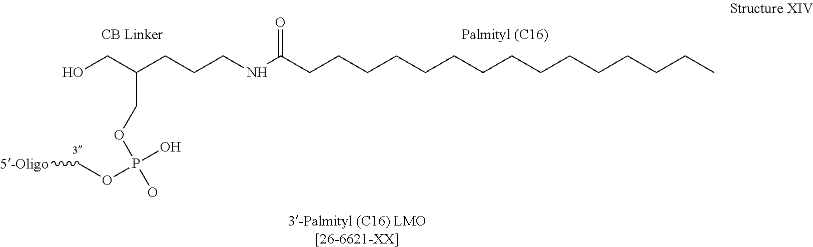

In some embodiments, the organic moiety can be attached to any nucleotide in the DNAzyme molecule. In some embodiments, the organic moiety is attached to the 3′ terminal nucleotide of the DNAzyme. In some embodiments, the organic moiety is attached to the 5′ terminal nucleotide of the DNAzyme. In some embodiments, an internal conjugate may be attached directly or indirectly through a linker to a nucleotide at a 2′ position of the ribose group, or to another suitable position.

In some embodiments, the organic moiety can be attached to any nucleotide within the DNAzyme molecule as long as the silencing activity (catalytic activity) of the DNAzyme is not compromised.

In some embodiments, coupling of the DNAzyme to an organic moiety can be carried out using standard procedures in organic synthesis. The skilled person will appreciate that the exact steps of the synthesis will depend on the exact structure of the molecule which has to be synthesized. For instance, if the molecule is attached to the organic moiety through its 5′ end, then the synthesis is usually carried out by contacting an amino-activated oligonucleotide and a reactive activated organic moiety.

According to one embodiment, the DNAzyme is coupled to an organic moiety and to a protecting group (e.g., the organic moiety is coupled to the 5′ end of the DNAzyme, and the protecting group to the 3′ end, or vice versa).

### DNAzymes

In some embodiments, a DNAzyme targets a RNA molecule. In some embodiments, the molecule is an RNA transcript.

In some embodiments, a DNAzyme comprises, in 5′ to 3′ order: (i) a first substrate-binding domain (also referred to herein as the “5′ arm”) comprising a sequence that base pairs with a first region of the RNA transcript; (ii) a DNAzyme catalytic core; and (iii) a second substrate-binding domain (also referred to herein as the “3′ arm”) comprising a sequence that base pairs with a second region of the RNA transcript positioned 5′ to the first region of the RNA transcript. In related embodiments, upon binding of the DNAzyme to the RNA transcript, the DNAzyme catalytic core cleaves the RNA transcript.

In some embodiments, the target molecule comprises regions that hybridize with the binding arms of the DNAzyme. In certain embodiments, the target regions are fully complementary with the binding arms of the DNAzyme. In other embodiments, the binding regions are not fully complementary with the binding arms of the DNAzyme, provided that they hybridize sufficiently with the DNAzyme such that the DNAzyme catalytic activity is not adversely affected. In some embodiments, the regions exhibit at least about 70%, 80%, 85%, 90%, or 95% complementarity. In some embodiments, the cleavage site is within a region of the substrate situated between the binding regions. In related embodiments, the terminal 5′- and 3′ ends of the cleavage site are each linked to a binding region at the appropriate corresponding terminus (e.g. 5′ to 3′) of the binding arm.

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in some embodiments, binding with specificity by substrate binding domains of a DNAzyme, such that the catalytic domain of the DNAzyme is brought in to close enough proximity with a target sequence to permit catalytic cleavage of the target sequence. The degree of complementarily between the substrate binding domains of the DNAzyme and the target region of a RNA (e.g. a resistant gene mRNA) can vary, but no more than by what is required in order to permit the DNAzyme to cleave or mediate cleavage (e.g. by RNase H) of the target region. Determination of binding free energies for nucleic acid molecules to determine percent complementarily is known in the art.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5. 6, 7, 8, 9, 10 out of 10 being 50%, 60%, to 70%, 80%, 90%, and 100% complementary, respectively). The terms “100% complementary”. “fully complementary”, and “perfectly complementary” indicate that all of the contiguous residues of a nucleic acid sequence can hydrogen bond with the same number of contiguous residues in a second nucleic acid sequence.

The DNAzyme additionally comprises a catalytic domain (also referred to as catalytic core) between the binding arms, generally in the form of a loop, which includes single-stranded DNA, and may optionally include double-stranded regions. The terminal 5′- and 3′ ends of the catalytic domain are each linked to a binding arm at the appropriate corresponding terminus of the binding arm. The catalytic region may incorporate modified nucleotides, including modified bases, backbone, sugars and/or linkages to the extent that such modifications do not have an adverse effect on catalytic activity (cleavage activity) of the DNAzyme.

According to one embodiment, the DNAzyme is a 10-23 type DNAzyme (i.e., comprises the 10-23 catalytic core). The term “10-23” refers to a general DNAzyme model. DNAzymes of the 10-23 model typically have a catalytic domain of 15 nucleotides, which are flanked by two substrate binding domains. The catalytic domain of 10-23 DNAzymes typically comprises the sequence ggctagctacaacga (SEQ ID NO: 28). The DNAzyme 10-23 typically cleaves mRNA strands that contain an unpaired purine-pyrimidine pair, in some cases at a position between the first and second region of the RNA transcript. The length of the substrate binding domains of 10-23 DNAzymes is variable and may be of either equal length or variable length. According to one embodiment, the length of the substrate binding domains ranges between 6 and 14 nucleotides. In some embodiments, the length is between 8-12 nucleotides. In some embodiments, the lengths of the arms are independent selected from 7, 8, 9, 10, 11, and 12.

In some embodiments, the DNAzyme is an 8-17 type DNAzyme. In some embodiments, the DNAzyme comprises the 8-17 catalytic core. The term “8-17” refers to a general DNAzyme model. DNAzymes of the 8-17 model typically have a catalytic domain of 14 nucleotides, which are flanked by two substrate binding domains. The catalytic domain of 8-17 DNAzymes typically comprises the sequence TCCGAGCCGGACGA (SEQ ID NO: 29). The length of the substrate binding domains of 8-17 DNAzymes is variable and may be of either equal length or variable length. According to one embodiment, the length of the substrate binding domains ranges between 6 and 14 nucleotides, e.g. between 8 and 12 nucleotides (e.g. 7, 8, 9, 10, 11, 12 nucleotides, e.g. 8 nucleotides).

In related aspects, the DNAzyme comprises modified nucleotides. In further related aspects, modified nucleotides comprise 2′-FANA.

In some embodiments, the DNAzyme molecule is selected from DNAzyme KPC-337, set forth in SEQ ID NO: 1; SHV-1-133 DNAzyme, set forth in SEQ ID NO: 3 or 4; In some embodiments, the DNAzyme molecule is DNAzyme TEM-588 comprising a nucleic acid sequence, set forth in SEQ ID NO: 4; USA300HOU-2333-1302 DNAzyme, set forth in SEQ NO: 5, 9-10, 18-19, or 23; mecA-650 DNAzyme, set forth in SEQ ID NO: 6-8, 13-15, 20 or 26; DNAzyme USA300HOU-2396-437, set forth in SEQ ID NO: 9, 10 or 23; mecA-647 DNAzyme, set forth in SEQ ID NO: 11 or 12; glpT-1122 DNAzyme, set forth in SEQ ID NO: 16 or 17; mecR1-146 DNAzyme, set forth in SEQ ID NO: 21; mecA-353 DNAzyme, set forth in SEQ ID NO: 22; USA300HOU-2333-676 DNAzyme, set forth in SEQ ID NO: 24; femA-545 DNAzyme; set forth in SEQ ID NO: 25; KPC-568 DNAzyme, set forth in SEQ ID NO: 31; KPC-36 DNAzyme, set forth in SEQ ID NO: 32; KPC-470 DNAzyme, set forth in SEQ ID NO: 33; or KPC-389 DNAzyme, set forth in SEQ ID NO: 34.

In some embodiments, the DNAzyme molecule is selected from DNAzyme KPC-563, comprising a nucleic acid sequence set forth in SEQ ID NO: 35. In some embodiments, the DNAzyme molecule is DNAzyme KPC-574, comprising a nucleic acid sequence set forth in SEQ ID NO: 37. In some embodiments, the DNAzyme molecule is DNAzyme KPC-633, comprising a nucleic acid sequence set forth in SEQ ID NO: 38. In some embodiments, the DNAzyme molecule is DNAzyme KPC-344, comprising a nucleic acid sequence set forth in SEQ ID NO: 39. In some embodiments, the DNAzyme molecule is DNAzyme OXA-18-294, comprising a nucleic acid sequence set forth in SEQ ID NO: 40. In some embodiments, the DNAzyme molecule is DNAzyme OXA-18-59, comprising a nucleic acid sequence set forth in SEQ ID NO: 41. In some embodiments, the DNAzyme molecule is DNAzyme OXA-18-125, comprising a nucleic acid sequence set forth in SEQ ID NO: 42. In some embodiments, the DNAzyme molecule is DNAzyme OXA-18-225, comprising a nucleic acid sequence set forth in SEQ ID NO: 43. In some embodiments, the DNAzyme molecule is DNAzyme SHV-1-127, comprising a nucleic acid sequence set forth in SEQ ID NO: 44. In some embodiments, the DNAzyme molecule is DNAzyme SHY-1-33, comprising a nucleic acid sequence set forth in SEQ ID NO: 45. In some embodiments, the DNAzyme molecule is DNAzyme SHV-1-197, comprising a nucleic acid sequence set forth in SEQ ID NO: 46. In some embodiments, the DNAzyme molecule is DNAzyme TEM-518, comprising a nucleic acid sequence set forth in SEQ ID NO: 47. In some embodiments, the DNAzyme molecule is DNAzyme, TEM-810, comprising a nucleic acid sequence set forth in SEQ ID NO: 48. In some embodiments, the DNAzyme molecule is DNAzyme TEM-14, comprising a nucleic acid sequence set forth in SEQ ID NO: 49.

In some embodiments, the above sequences are modified. In some embodiments, the modification comprises the addition of 1-10 nucleotides, in other embodiments 1-8 nucleotides, in other embodiments 1-6 nucleotides, in other embodiments 1-5 nucleotides, in other embodiments 1-4 nucleotides, in other embodiments 1-3 nucleotides, or in other embodiments 1-2 nucleotides to the external ends of one or both substrate binding domains (binding arms) of the DNAzyme. In some embodiments, the additions are overhangs that are non-complementary to the target.

As provided herein, 45 conjugated DNAzymes capable of targeting various bacterial resistance genes were tested, including KPC, SHV-1, TEM, USA300HOU, mecA, mecR1, OXA-18, glpT and FemA (see Tables 1 and 2, and Example 1, hereinbelow). For example, a DNAzyme termed KPC-337 (as set forth in SEQ ID NO: 1) was designed to target a conserved region in RNA transcripts of the gene bla carbapenemase of clinically relevant strains of Klebsiella pneumoniae (Example 2, hereinbelow), KPC-337 was capable of entering the bacteria and binding to and specifically cleaving the intracellular RNA transcripts of bla carbapenemase (Example 2, hereinbelow). Furthermore, the combined treatment of KPC-337 with sub-toxic concentrations of meropenem was bactericidal, thus illustrating that potentiation of the antibiotic was achieved (Example 2). The bactericidal effect of conjugated DNAzyme treatment with sub-toxic levels of meropenem on KPC-337 was further illustrated in an ex-vivo model of lung disease (Example 3) and in in vivo infection larvae and murine models (Example 4). The KPC-337 was not toxic to human cells at bioactive concentration (Example 4).

Furthermore, cholesterol-conjugated DNAzymes targeting the resistance genes mecA, mecR1, glpT, femA and the efflux pump USA300HOU_2333 (DNAzyme sequences set forth in SEQ ID NO: 5-26) were designed to target a conserved region in their corresponding RNA transcripts in MRSA (Example 6, hereinbelow). These conjugated DNAzymes were all demonstrated to be capable of entering the bacteria. Furthermore, the combined use of each of these DNAzymes with the antibiotic cefoxitin inhibited bacterial growth. Thus, the use of conjugated DNAzymes increased bacterial susceptibility to antibiotic treatment (Example 6).

Those skilled in the art will understand that the described conjugated DNAzymes comprise nucleotides. The terms “nucleotide” “nucleobase” and the like may encompass various types of nucleotides, either deoxyribonucleotides or ribonucleotides, or analogs thereof, comprising various nucleobases. In some embodiments, a nucleotide or nucleobase includes a naturally occurring purine or pyrimidine base found in DNA (e.g., an adenine “A”, a guanine “G”, a thymine “T”, or a cytosine “C”) or a naturally occurring purine or pyrimidine base found in RNA (e.g., an adenine “A”, a guanine “G”, an uracil “U” or a cytosine “C”). In other embodiments, a nucleotide or nucleobase includes nucleic acids derived from synthetic polynucleotide and/or oligonucleotide molecules composed of naturally occurring bases, sugars, and covalent internucleoside linkages (e.g., backbone). In other embodiments, a nucleotide or nucleobase synthetic polynucleotides and/or oligonucleotides having non-naturally occurring portions, which function similarly to respective naturally occurring portions.

Various modifications to DNAzyme molecules can be made to enhance the utility of these molecules. Such modifications can enhance affinity for the nucleic acid target, increase activity, increase specificity, increase stability and decrease degradation (e.g., in the presence of nucleases), increase shelf-life, enhance half-life and/or improve introduction of such DNAzyme molecules to the target site (for example, to enhance penetration of cellular membranes, confer the ability to recognize and bind to targeted cells, and enhance cellular uptake).

In certain embodiments, modified bases (which may be, for example, non-naturally occurring bases) preserving the base pair specificity of the parent DNA or RNA base are considered equivalent to the DNA or RNA parent bases, e.g., a sequence mentioned herein as containing “guanine” contain instead modified forms of guanine preserving the base pair specificity of guanine.

In some embodiments, the described DNAzymes comprise one or more chemical modifications. In some embodiments, the one or more chemical modifications are selected from the group consisting of base modifications, sugar modifications, and inter-nucleotide linkage modifications. In some embodiments, the one or more chemical modifications are selected from the group consisting of locked nucleic acids (LNA), phosphorothioate, 2-O-fluoro, 2-O-methyl, 2-O-methoxyethyl, phosphoramidate morpholino, and methyl-cytosine.

Non-limiting examples of modifications are provided in Table 1.

TABLE 1
Embodiments of chemical modifications.

| Terminal | Sugar ring | Nitrogen base | Backbone |
|---|---|---|---|
| Biotin | 2'-OH | 5'-BzdU | Phosphorothioate |
| | (RNA) | | |
| Inverted-dT | 2'-OMe | Naphtyl | Methylphos- |
| | | | phorothioate |
| PEG (0.5-40 kDa) | 2'-F | Triptamino | Phosphorodithioate |
| Cholesterol | 2'-NH2 | Isobutyl | Triazole |
| Albumin | LNA | 5-Methyl | Amide (PNA) |
| | | Cytosine | |
| Chitin (0.5-40 kDa) | UNA | Alkyne | Alkyne |
| | | (dibenzo- | (dibenzo- |
| | | cyclooctyne) | cyclooctyne) |
| Chitosan (0.5-40 kDa) | 2'-F ANA | Azide | Azide |
| Cellulose (0.5-40 kDa) | L-DNA | Maleimide | Maleimide |
| Terminal amine | CeNA | | |
| (alkyne | | | |
| chain with amine) | | | |
| Alkyl | TNA | | |
| (dibenzocyclooctyne) | | | |
| Azide | HNA | | |
| Thiol | | | |
| Maleimide | | | |
| N-hydroxysuccinimide | | | |
| nnnnnnnn | | | |

In some embodiments, the described DNAzymes comprise a 5′ end cap. In some embodiments, the 5′ end cap comprises an inverted thymidine, terminal amine, alkyne, azide, thiol, maleimide, or N-hydroxysuccinimide. In certain embodiments, the DNAzymes comprise a 3′ end cap. In some embodiments, the 3′ end cap comprises an inverted thymidine; or bases modified with terminal amine, alkyne, azide, thiol, maleimide, or N-hydroxysuccinimide.

In certain embodiments, the described DNAzymes comprise one or more modified sugars. In some embodiments, the described DNAzymes comprise one or more 2′ sugar substitutions (e.g., a 2′-fluoro, a 2′-amino, or a 2′-O-methyl substitution). In certain embodiments, the DNAzymes to comprise locked nucleic acid (LNA), unlocked nucleic acid (UNA) and/or 2′deozy-2′fluoro-D-arabinonucleic acid (2′-FANA) sugars in their backbone.

In certain embodiments, the described DNAzymes comprise one or more methylphosphonate internucleotide bonds and/or a phosphorothioate (PS) internucleotide bonds. In certain embodiments, the described DNAzymes comprise one or more (e.g., at least 1, 2, 3, 4, 5, 6, 7, 8, 9, 10 11, 12, 13, 14, 15, 16, 17, 18, 19, 20, 21, 22, 23, 24, 25, 26, 27, 28, 29, 30 31, 32, 33, 34, or 35) triazole internucleotide bonds. In certain embodiments, the described DNAzymes are modified with a cholesterol or a dialkyl lipid (e.g., on their 5′ end, 3′ end, or both ends).

In some embodiments, the described DNAzymes comprise one or more modified bases (e.g., 5-(N-benzylcarboxyamide)-2′-deoxyuridine) [5-BzdU], beta-naphthyl-, tryptamine, or isobutyl substituted bases; 5-methyl cytosine, or bases modified with alkyne, dibenzocyclooctyne, azide, or maleimide).

In some embodiments, the modified bases comprise one or more of 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil, 8-halo; 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. In other embodiments, the modified bases comprise one or more of 5-substituted pyrimidines, 6-azapyrimidines, and N-2, N-6, and O-6-substituted purines, including 2-aminopropyladenine, 5-propynyluracii, and 5-propynylcytosine. In other embodiments, the modified bases comprise 5-methylcytosine substitutions combined with 2′-O-methoxyethyl sugar modifications.

In certain embodiments, the described DNAzymes are DNA DNAzymes (e.g., D-DNA DNAzymes or enantiomer L-DNA DNAzymes). In other embodiments, the described DNAzymes are RNA DNAzymes (e.g., D-RNA DNAzymes or enantiomer L-RNA DNAzymes). In other embodiments, the described DNAzymes comprise a mixture of DNA and RNA.

### Target Bacteria

The term “bacteria” as used herein generally refers to a genus of prokaryotic microorganisms scientifically classified as such. Most bacteria can be classified as Gram-positive bacteria or Gram-negative bacteria.

Gram-positive bacteria relate to bacteria encapsulated by a single lipid bilayer (membrane) and a thick layer (20-80 nm) of peptidoglycan, which retains the crystal violet stain in a Gram staining technique. Exemplary Gram-positive bacteria include, but are not limited to, Actinomyces israelli, Bacillus species, Bacillus antracis, Brevibacillus, Clostridium, Clostridium perfringens, Clostridium tetani, Cornyebacterium, Corynebacterium diphtheriae, Enterococcus (e.g. Enterococcus faecum), Erysipelothrix rhusiopathiae, Lactobacillus, Listeria, Mycobacterium, Staphylococcus (e.g. Staphylococcus aureus), Streptomyces and Streptococcus.

Gram-negative bacteria relate to bacteria encapsulated by a double lipid bilayer (inner and outer cell membranes) with a relatively thin layer of peptidoglycan between the two membranes; which is unable to retain crystal violet stain in a Gram staining technique. Exemplary Gram-negative bacteria include, but are not limited to, Aerobacter, Aeromonas, Acinetobacter (e.g. Acinetobacter baumannii), Agrobacterium, Bacteroides, Bartonella, Bordetella, Borrelia, Brucella, Burkholderia, Campylobacter, Calymmatobacterium, Campylobacter, Capnocytophaga, Cardiobacterium, Citrobacter, Chlamydia, Chlamydophila, Eikenella, Enterobacter, Enterobacter aerogenes, Escherichia, Flavobacterium, Francisella, Fusobacterium, Fusobacterium nucleatum, Gardnerella, Haemophilus, Hafnia, Helicobacter, Kingella, Klebsiella (e.g. Klebsiella pneumoniae), Legionella, Leptospira, Morganella, Moraxella, Mycoplasma, Neisseria, Pasteurella (e.g. Pasteurella multocida), Plesiomonas, Prevotella, Proteus, Providencia, Pseudomonas (e.g. Pseudomonas aeruginosa), Porphyromonas, Rickettsia, Salmonella, Serratia, Shigella, Stenotrophomonas, Streptobacillus, Streptobacillus moniliformis, Stenotrophomonas, Spirillum, Treponema (e.g., Treponema pallidium, Treponema pertenue), Xanthomonas, Veillonella, Vibrio, and Yersinia.

Additional bacterial species, e.g., which are neither gram-positive or gram-negative, include, but are not limited to, Borelia.

According to one embodiment, the bacteria are pathogenic bacteria.

According to one embodiment, the bacteria cause a nosocomial infection.

According to one embodiment, the bacteria are resistant to an antimicrobial treatment, such as to an antibiotic. Exemplary antibiotics include, but are not limited to, beta-lactam antibiotics such as penicillin, methicillin, oxacillin, cephalosporin (e.g., third-generation oxyimino-cephalosporins e.g., ceftazidime, cefotaxime, and ceftriaxone; or methoxy-cephalosporins, e.g., cephamycin and carbapenem), cefoxitin, cefamandole, cefoperazone, imipenem, meropenem, aztreonam; macrolide antibiotics such as erythromycin, erythromycin thiocyanate; aminoglycoside antibiotics such as streptomycin, kanarnycin, neomycin; tetracycline antibiotics such as minocycline, doxycycline; fluoroquinolone antibiotics such as ciprofloxacin, gemifloxacin, levofloxacin, moxifloxacin, ofloxacin; and polypeptide antibiotics such as vancomycin. According to one embodiment, the bacteria are resistant to multiple antimicrobial treatments (i.e., multidrug resistant (MDR)).

In some embodiments, the bacterium is an Enterococcus faecium, a Staphylococcus aureus, a Klebsiella pneumoniae, an Acinetobacter baumannii, a Pseudomonas aeruginosa or an Enterobacter.

In some embodiments, the bacterium is Klebsiella pneumoniae (also referred to as K. pneumoniae).

In some embodiments, the bacterium is Staphylococcus aureus.

In some embodiments, the bacterium is methicillin-resistant Staphylococcus aureus (MRSA).

In some embodiments, the bacterium is Pseudomonas aeruginosa.

According to one embodiment, the phrase “render the bacteria susceptible to antibiotic treatment” refers to increasing susceptibility of the bacteria such that the bacteria are more susceptible to an antibiotic treatment by at least about 10%, 20%, 30%, 40%, 50%, 60%, 70%, 80%, 90% or 100% as compared to bacteria not treated by the conjugated DNAzyme.

Bacteria which are more susceptible to treatment will typically exhibit suspension of growth and cell death (bactericidal effect), upon treatment with antibiotics. Methods for determining growth or death of bacteria are well known in the art. By way of example, the quantity of a target bacterial species or strain can be determined by growth in a culture, such as a liquid culture. In this regard, as the bacteria multiply and increase in number, the optical density of the liquid culture increases (due to the presence of an increasing number of bacterial cells). Thus, an increase in optical density indicates bacterial growth while a decrease in optical density indicates a decline in bacterial growth and bacterial death. For example, optical density (at for example at 600 nm) can be determined within the wells of a multi-well plate (e.g. a 96-well plate) using an automated plate reader.

According to one embodiment, the target gene of a bacteria is a resistance gene.

As used herein, the term “resistance gene” or “antibiotic resistance gene” can interchangeably be used to refer to a gene conferring antibiotic resistance in bacteria.

In some embodiments, the resistance gene can be either a genomic or a plasmid gene, or from other sources such as genetic vectors acquired or engineered into the bacteria.

In some embodiments, the bacterial target gene is the RNA transcript of a bacterial resistance gene.

The antibiotics targeted by the resistance gene can be, for example, beta-lactams, macrolides, aminoglycosides, tetracyclines, fluoroquinolones and polypeptide antibiotics (as discussed in detail above).

According to one embodiment, the resistance gene confers beta-lactam antibiotic resistance.

In some embodiments, the resistance gene confers carbapenem antibiotic resistance. In some embodiments, the resistance gene confers penicillin antibiotic resistance. In some embodiments, the resistance gene confers cephalosporin antibiotic resistance.

In some embodiments, the resistance gene confers monobactam antibiotic resistance.

According to one embodiment, when the bacteria are resistant to multiple antimicrobial treatments, such as in cases of mixed infection, one or more DNAzymes can be used for different targets.

### Pharmaceutical Compositions

In certain embodiments, provided herein are pharmaceutical compositions comprising a conjugated DNAzyme. In some embodiments, a pharmaceutical composition comprises a therapeutically effective amount of a conjugated DNAzyme.

A skilled artisan would recognize that an “effective amount” (or, “therapeutically effective amount”) may encompass an amount sufficient to effect a beneficial or desired clinical result upon treatment. An effective amount can be administered to a subject in one or more doses. In terms of to treatment, an effective amount is an amount that is sufficient to palliate, ameliorate, stabilize, reverse or slow the progression of the disease, or otherwise reduce the pathological consequences of the disease, for example but not limited to bacterial infections and sequelae thereof. The effective amount is generally determined by the physician on a case-by-case basis and is within the skill of one in the art. Several factors are typically taken into account when determining an appropriate dosage to achieve an effective amount. These factors include age, sex and weight of the subject, the condition being treated, the severity of the condition and the form and effective concentration of the antigen-binding fragment administered.

In some embodiments, the pharmaceutical composition comprises a plurality of conjugated DNAzymes. In some embodiments, the pharmaceutical composition comprises 2, 3, 4, 5, 6, 7, 8, 9, 10, 20, or more DNAzymes described herein. In some embodiments, the pharmaceutical composition comprising a plurality of DNAzymes, comprises equal amounts of different DNAzymes. In some embodiments, the pharmaceutical composition comprises a plurality of DNAzymes at varying ratios.

Formulation of the pharmaceutical composition may be adjusted according to applications. In particular, the pharmaceutical composition may be formulated using a method known in the art to provide rapid, continuous, or delayed release of the active ingredient after administration to mammals.

In some embodiments, the pharmaceutical composition is in a form of a sterile injectable solution.

In some embodiments, the pharmaceutical composition is suitable for administration via a route selected from the group consisting of intramuscular, subcutaneous, intravenous, intraperitoneal, inhaled, intranasal, intraarterial, intravesical, and intraocular.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives; gelatin, vegetable oils and polyethylene glycols.

Other carriers or excipients which may be used include, but are not limited to, materials derived from animal or vegetable proteins, such as the gelatins, dextrins and soy, wheat and psyllium seed proteins; gums such as acacia, guar, agar, and xanthan; polysaccharides; alginates; carboxymethylcelluloses; carrageenans; dextrans; pectins; synthetic polymers such as polyvinylpyrrolidone; polypeptide/protein or polysaccharide complexes such as gelatin-acacia complexes; sugars such as mannitol, dextrose, galactose and trehalose; cyclic sugars such as cyclodextrin; inorganic salts such as sodium phosphate, sodium chloride and aluminium silicates; and amino acids having from 2-12 carbon atoms and derivatives thereof such as, but not limited to, glycine, L-alanine, L-aspartic acid, L-glutainic acid, L-hydroxyproline, L-isoleucine, L-leucine and L-phenylalanine.

Solutions or suspensions used for parenteral, intradermal, or subcutaneous application typically include the following components: a sterile diluent such as water for injection, saline solution, fixed oils, polyethylene glycols, glycerine, propylene glycol (or other synthetic solvents), antibacterial agents (e.g., benzyl alcohol, methyl parabens), antioxidants (e.g., ascorbic acid, sodium bisulfite), chelating agents (e.g., ethylenediaminetetraacetic acid), buffers (e.g., acetates, citrates, phosphates), and agents that adjust tonicity (e.g., sodium chloride, dextrose). The pH can be adjusted with acids or bases, such as hydrochloric acid or sodium hydroxide, for example. The parenteral preparation can be enclosed in ampules, disposable syringes or multiple dose glass or plastic vials.

Pharmaceutical compositions adapted for parenteral administration include, but are not limited to, aqueous and non-aqueous sterile injectable solutions or suspensions, which can contain antioxidants, buffers, and solutes that render the compositions substantially isotonic with the blood of an intended recipient. Extemporaneous injection solutions and suspensions can be prepared from sterile powders, granules and tablets. Such compositions may comprise a therapeutically effective amount of a conjugated DNAzyme and/or other therapeutic agent(s), together with a suitable amount of carrier to provide the form for proper administration to the subject.

In some embodiments, the conjugated DNAzyme is encapsulated in a liposome, conjugated to a micro- or nano-particle, or embedded in a polymer matrix such as gel, PLGA, PEG, etc.

Lipid-based systems which may be used for encapsulating DNAzyme molecules for in-vivo administration to a subject, who may have a bacterial infection. Lipid-based systems include, for example, liposomes, lipoplexes and lipid nanoparticles (LNPs).

Liposomes include any synthetic (i.e., not naturally occurring) structure composed of lipid bilayers, which enclose a volume. Liposomes may be in the form of emulsions, foams, micelles, insoluble monolayers, liquid crystals, or phospholipid dispersions.

In some embodiments, for in vivo therapy, the composition of matter comprising a DNAzyme molecule is administered to the subject per se or as part of a pharmaceutical composition.

According to another aspect, there is provided a pharmaceutical composition, comprising at least one conjugated DNAzyme. In some embodiments, the composition is comprised of 1 (one) DNAzyme, 2 (two) DNAzymes, or more in varying ratios.

In some embodiments of the pharmaceutical composition of matter, the composition is comprised of at least one DNAzyme and at least one antibiotic compound.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient(s)” refers to the DNAzyme molecule and, if present, the antibiotic.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

In some embodiments of the pharmaceutical composition, the composition includes a combination of 1, 2, or more DNAzymes.

In some embodiments of the pharmaceutical composition, the composition includes a combination of 1, 2, or more DNAzymes, and an antibiotic compound such as penicillin, methicillin, oxacillin, cefoxitin, cephalosporin, carbapenem, imipenem, meropenem, aztreonam, etc.

In some embodiments, compositions are presented in a pack or dispenser device, such as an FDA approved kit, which may contain one or more unit dosage forms containing the active ingredient. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation formulated in a compatible pharmaceutical carrier may also be prepared, placed in an appropriate container, and labeled for treatment of an indicated condition, as is further detailed above.

According to one embodiment, there is provided an article of manufacture comprising a described conjugated DNAzyme, being packaged in a packaging material and identified in print, in or on the packaging material for use in the treatment of a bacterial infection.

In some embodiments, the described therapeutic compositions may comprise, in addition to the conjugated DNAzyme, other known medications for the treatment of bacterial infections, e.g., antibacterial agents such as antibiotics. Exemplary antibiotics which can be used in accordance with some embodiments include, but are not limited to, penicillins (e.g., oxacillin, methicillin, amoxicillin and amoxicillin-clavulanate), monobactams (e.g., aztreonam), clavulanate acid, trimethoprim-sulfamethoxazole, cephalosporins (e.g., third-generation oxyimino-cephalosporins and methoxy-cephalosporins, including but not limited to, ceftazidime, cefotaxime, cefuroxime, ceflacor, cefprozil, loracarbef, cefindir, cefixime, cefpodoxime proxetil, ceflbuten, ceftriaxone, cephamycins (e.g., cefoxitin), carbapenems (e.g., imipenem-cilastatin, meropenem, ertapenem, doripenem, panipenem-betamipron, and biapenem)), fluoroquinolone (e.g., ofloxacin, ciprofloxacin, levofloxacin, trovafloxacin), macrolides, azalides (e.g., erythromycin, clarithromycin, and azithromycin), sulfonamides, ampicillin, tetracycline, chloramphenicol, minocycline, doxycycline, vancomycin, bacitracin, kanamycin, neomycin, gentamycin, erythromycin, spectinomycin, zeomycin, streptomycin as well as any of combinations and any derivatives thereof.

In some embodiments, the antibiotic is a beta-lactam.

In some embodiments, the antibiotic is a Carbapenem.

In some embodiments, the antibiotic is a Penicillin.

In some embodiments, the antibiotic is a Cephalosporin.

In some embodiments, the antibiotic is a Monobactam.

In some embodiments, the DNAzyme and antibiotic in any of the above described compositions are packaged together.

In other embodiments, the DNAzyme and antibiotic in any of the above described compositions may be packaged separately.

As mentioned above, the described DNAzyme is capable of increasing susceptibility of bacteria to antibiotic therapy. Accordingly, the DNAzymes may be used alone, or with antibiotics, to treat bacterial infections.

According to another aspect, there is provided a method of treating a bacterial infection in a subject in a need thereof, comprising administering to the subject the described pharmaceutical composition.

As used herein, the term “subject” or “subject in need thereof” includes mammals, such as human beings, male or female; at any age which suffers from a bacterial infection.

In some embodiments, the subject is a human subject.

In some embodiments, the subject is a non-human subject (e.g., a farm animal, e.g., a mammal including e.g., a horse, a donkey, a pig, a sheep, a goat, a cow, a dog, a cat, a rabbit, a rat, a hamster, a mouse; a chicken, a duck, a goose).

According to one embodiment, the bacterial infection is caused by gram-negative bacteria.

According to one embodiment, the bacterial infection is caused by gram-positive bacteria.

According to one embodiment, the bacterial infection is caused by a single microbial species.

According to one embodiment, the bacterial infection is caused by two or more bacterial species).

According to one embodiment, the bacterial infection is caused by an antibiotic-resistant bacterial strain.

In some embodiments, the bacterial infection is caused by an Enterococcus faecium, a Staphylococcus aureus, a Klebsiella pneumoniae, an Acinetobacter baumannii, a Pseudomonas aeruginosa or an Enterobacter.

In some embodiments, the bacterial infection is caused by Klebsiella pneumoniae.

In some embodiments, the bacterial infection is caused by Staphylococcus aureus.

In some embodiments, the bacterial infection is caused by methicillin-resistant Staphylococcus aureus (MRSA).

In some embodiments, the bacterium is Pseudomonas aeruginosa.

Non-limiting examples of diseases and disorders caused by bacteria that may, be treated by the described methods include, but are not limited to, actinomycosis, anaplasmosis, anthrax, bacillary angiomatosis, actinomycetoma, bacterial pneumonia, bacterial vaginosis, bacterial endocarditis, bartonellosis, botulism, boutenneuse fever, brucellosis, bejel, brucellosis spondylitis, bubonic plague, Buruli ulcer, Bairnsdale ulcer, bacillary dysentery, campylobacteriosis, Carrion's disease, cellulitis, chancroid, Chlamydia infection, Chlamydia pneumonia, Chlamydia conjunctivitis, clostridial myonecrosis, cholera, Clostridium difficile colitis, diphtheria, Daintree ulcer, donavanosis, dysentery, erhlichiosis, epidemic typhus, Far East scarlet-like fever, glanders, gonorrhea, granuloma inguinale, human necrobacillosis, hemolytic-uremic syndrome, human ewingii ehrlichiosis, human monocytic ehrlichiosis, human granulocytic anaplasmosis, infant botulism, Izumi fever, Kawasaki disease, Kumusi ulder, lymphogranuloma venereum, Lemierre's syndrome, Legionellosis, leprosy, leptospirosis, listeriosis, Lyme disease, lymphogranuloma venereum, Malta fever, Mediterranean fever, myonecrosis, mycoburuli ulcer, mucocutaneous lymph node syndrome, meliodosis, meningococcal disease, murine typhus, Mycoplasma pneumonia, mycetoma, neonatal conjunctivitis, nocardiosis, Oroya fever, ophthalmia neonatorum, ornithosis, Pontiac fever, peliosis hepatis, pneumonic plague, postanginal shock including sepsis, pasteurellosis, pelvic inflammatory disease, pertussis, pneumococcal infection, pneumonia, psittacosis, parrot fever, pseudotuberculosis, Q fever, quintan fever, rabbit fever, rickettsialpox, Rocky Mountain spotted fever, Reiter syndrome, rheumatic fever, salmonellosis, scarlet fever, sepsis, septicemic plague, Searls ulcer, shigellosis, soft chancre, syphilis, streptobacillary fever, scrub typhus, trachoma, tuberculosis, tularemia, typhoid fever, typhus, tetanus, toxic shock syndrome, undulant fever, ulcus molle, Vibrio parahaemolyticus enteritis, Whitmore's disease, Waterhouse-Friderichsen syndrome, yaws, and yersiniosis.

It is within the skill of the art to select a physiologically acceptable steroid for a given subject.

In certain embodiments, the described conjugated DNAzyme is administered to a subject in need together with an antibiotic.

According to one embodiment, the antibiotic and the conjugated DNAzyme are in separate formulations.

According to one embodiment, the antibiotic and the conjugated DNAzyme are in a co-formulation.

According to one embodiment, an efficient anti-bacterial treatment is determined when there is a decrease of at least about 10%, 20%, 30%, 40%, 50%, 60%, 70%, 80%, 90% or 100% or more in the number of bacterial cells or bacterial products (e. g., toxins), as compared to the number of bacterial cells or products in the subject being treated but prior to the treatment.

Throughout this application, various embodiments may be presented in a range format. It should be understood that the description in range format is merely for convenience and brevity is and should not be construed as an inflexible limitation on the described scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the embodiments in a non-limiting fashion.

### General Materials and Experimental Procedures

### DNAzymes

DNAzymes, including any modification, were ordered from Integrated DNA Technologies (IDT), reconstituted to 100 μM with ultrapure, DNase/RNase free water (Biological Industries, Israel), and stored at −20° C. DNA sequences are listed in Tables 2 and 3, below.

TABLE 2
DNAzymes effective against Klebsiella

| | | SEQ ID |
|---|---|---|
| DNAzyme | Sequence | NO |
| KPC_337 | GCCTGTTGggctagctacaacgaCAGATATTT/3CholTEG/ | 1 |
| KPC_332 | GTTGTCAGAggctagctacaacgaATTTTTCCG/3CholTEG/ | 2 |
| SHV-1_133 | CCAGATCCAggetagctacaacgaTTCTATCAT/3CholTEG/ | 3 |
| TEM_588 | TAGAGTAAGggctagctacaacgaAGTTCGCC/3CholTEG/ | 4 |
| KPC_568 | CAGTTTTTGggctagctacaacgaAAGCTTTCC/3CholTEG/ | 31 |
| KPC_36 | CATGAGAGAggctagctacaacgaAAGACAGCA/3CholTEG/ | 32 |
| KPC_470 | GAACGTGGggctagctacaacgaATCGCCGA/3CholTEG/ | 33 |
| KPC_389 | CGGCGTTAggctagctacaacgaCACTGTATT/3CholTEG/ | 34 |
| KPC_563 | TTTTGTAAGggctagctacaacgaTTTCCGTCA/3CholTEG/ | 35 |
| KPC_332 | GTTGTCAGAggctagctacaacgaATTTTTCCG/3CholTEG/ | 36 |
| KPC_574 | CAGTGTCAGggctagctacaacgaTTTTGTAAG/3CholTEG/ | 37 |
| KPC_633 | GTGTTTCCtccgagccggacgaTTAGCCAAT/3CholTEG/ | 38 |
| KPC_344 | CCGTCATGggctagctacaacgaCTGTTGTCA/3CholTEG/ | 39 |
| OXA-18_294 | ATAGACTTGggctagctacaacgaTTGTATGTG/3CholTEG/ | 40 |
| OXA-18_59 | CCACGGAAggctagctacaacgaTGATTGGGA/3CholTEG/ | 41 |
| OXA-18_125 | TATAAGGTAggctagctacaacgaTTCCGGTAA/3CholTEG/ | 42 |
| OXA-18_225 | TTTGGCGAggctagctacaacgaTGCAAGATT/3CholTEG/ | 43 |
| SHV-1_127 | CCATTTCTAggctagctacaacgaCATGCCTAC/3CholTEG/ | 44 |
| SHV-1_33 | GGTGGCTAAggctagctacaacgaAGGGAGATA/3CholTEG/ | 45 |
| SHV-1_197 | TTAAAGGTGggctagctacaacgaTCATCATGG/3CholTEG/ | 46 |
| TEM_518 | GCTCGTCGggctagctacaacgaTTGGTATGG/3CholTEG/ | 47 |
| TEM_810 | TCTATTTCGggctagctacaacgaTCATCCATA/3CholTEG/ | 48 |
| TEM_14 | ACACGGAAAggctagctacaacgaGTTGAATAC/3CholTEG/ | 49 |

TABLE 3
DNAzymes effective against MRSA

| | | SEQ |
|---|---|---|
| DNAzyme | Sequence | ID NO |
| USA300HOU_ | TTTTAGTTGggctagctacaacgaGTTAGTACT/3CholTEG/ | 5 |
| 2333_1302_9 nt | | |
| mecA_650_10 nt | TATTTTAGCAggctagctacaacgaAGTCATTTAA/3CholTEG/ | 6 |
| mecA_661_10 nt | TGACTCATAAggctagctacaacgaTATTTTAGCA/3CholTEG/ | 7 |
| mecA-658_11 nt | TGACTCATAATggctagctacaacgaTTTAGCATAGT/3CholTEG/ | 8 |
| USA300HOU_ | GCTTTTTTAggctagctacaacgaTGACTAATG/3CholTEG/ | 9 |
| 2396:_437_9 nt | | |
| USA300HOU_ | AGCTTTTTTAggctagctacaacgaTGACTAATGG/3CholTEG/ | 10 |
| 2396:_437_10 nt | | |
| mecA_647_9 nt | TTAGCATAGggctagctacaacgaCATTTAAAT/3CholTEG/ | 11 |
| mecA_647-11 nt | TTTTAGCATAGggctagctacaacgaCATTTAAATAA/3CholTEG/ | 12 |
| mecA_650_9 nt | ATTTTAGCAggctagctacaacgaAGTCATTTA/3ChoITEG/ | 13 |
| mecA_650_11 nt | TTATTTTAGCAggctagctacaacgaAGTCATTTAAA/3CholTEG/ | 14 |
| mecA_661_9 nt | GACTCATAAggctagctacaacgaTATTTTAGC/3CholTEG/ | 15 |
| glpT_1122_9 nt | TTTAGTAAGggctagctacaacgaTCTTTATGG/3CholTEG/ | 16 |
| glpT_1122_10 nt | ATTTAGTAAGggctagctacaacgaTTATGGGTAC/3CholTEG/ | 17 |
| USA300HOU_ | TTTTAGTTGggctagctacaacgaGTTAGTACT/3CholTEG/ | 18 |
| 2333_1302_9 nt | | |
| USA300HOU_ | TTTTTAGTTGggctagctacaacgaGTTAGTACTC/3CholTEG/ | 19 |
| 2333_1302_10 nt | | |
| mecA-658_9 nt | TCATAATTAggetagctacaacgaTTTAGCATA/3CholTEG/ | 20 |
| mecR1_146 | GAAGTCGTGggctagctacaacgaCAGATACAT/3CholTEG/ | 21 |
| mecA_353 | GTTCGTTGtccgagccggacgaCGAATAATT/3CholTEG/ | 22 |
| USA300HOU_ | GCTTTTTTAggctagctacaacgaTGACTAATG/3CholTEG/ | 23 |
| 2396:_437 | | |
| USA300HOU_ | ATTTATTTAggetagctacaacgaAAAATTTAC/3CholTEG/ | 24 |
| 2333:676 | | |
| femA_545 | TTTTTCGTGggctagctacaacgaTTCTTTTTC/3CholTEG/ | 25 |
| mecA_658_9 nt | TCATAATTAggctagctacaacgaTTTAGCATA/3CholTEG/ | 26 |

### Bacterial Strains

Klebsiella pneumoniae (KP, ATCC® BAA-1705™), Staphylococcus aureus (ATCC® BAA-1717™) and Pseudomonas aeruginosa (PA, ATCC® BAA-3105™) were purchased from ATCC, streaked onto Luria broth (LB) agar plates (HyLabs, Israel) and grown for 24 hours at 37° C. A single colony from each strain was arbitrarily selected and frozen in LB supplemented with 30% glycerol (HyLabs, Israel) and stored at −80° C. for all assays.

### Flow Cytometry

Flow cytometry was performed on a Becton-Dickinson Accuri™ C6 Plus cytometer equipped with 488 nm solid-state laser and a 640 nm diode laser. Data was analyzed using Kaluza Analysis 2.1 software using a C6 import module. To measure intracellular fluorescent DNAzyme concentration, bacterial strains were streaked from frozen LB+glycerol stocks onto LB agar plates and incubate for 24 hours at 37° C. Single colonies were grown overnight at 37° C. in 3 ml of LB. Cultures were diluted 1:100 in 2 ml LB and grown until mid-log (optical density [OD]₆₀₀ 0.6-0.8) at 37° C. Fluorescent DNAzyme were added to a final concentration of 1.25 μM. Cells were harvested every 30 minutes, washed twice with Dulbecco's Phosphate Buffered Saline (PBS, Biological Industries), treated with DNase I (New England Biolabs) for 5 min. in PBS at room temperature, and washed again with PBS. Data were collected from ˜50,000 cells per time point using the FL-4 laser.

### Growth Assays

Bacterial strains were streaked from frozen LB+glycerol stocks onto LB agar plates and incubated for 24 hours at 37° C. Single colonies were grown overnight at 37° C. in 3 ml of LB. Cultures were diluted 1:1000 in 2 ml LB+128 μg/ml meropenem, and DNAzyme was added to 1.25 μM. Cultures were incubated at 37° C. with continuous shaking and the OD₆₀₀ was recorded every 30 min, using Ultrospec™ 10 (Biochrom™). Viability was tested by serially diluting (1:100) bacterial suspensions and plating spots on LB plates. The plates were incubated at 37° C. overnight, and CFU was counted to determine culture viability.

### Bla-KPC Quantification From Bacterial Cells

Bacterial strains were streaked from frozen LB+glycerol stocks onto LB agar plates and incubate for 24 hours at 37° C. Single colonies were grown overnight at 37° C. in 3 ml of LB. Cultures were diluted 1:1000 in 2 ml LB+128 μg/ml meropenem, and DNAzyme was added to 1.25 μM. Cultures were incubated at 37° C. with continuous shaking for 20 min., then 1 ml culture was added to 1 ml RNAprotect® (Qiagen®). Total RNA was extracted from each sample using RNeasy® Mini Kit (Qiagen®). Total RNA concentration, purity, and integrity were determined using NANODROP™ and gel electrophoresis. cDNA synthesis was performed using iScript™ cDNA synthesis (Bio-Rad®). Equal amounts of total RNA (1000 ng/20 μL) were reverse-transcribed in all samples. Reactions were incubated in a CFX96 Touch™ Real-Time PCR detection system (Bio-Rad®) by the following program: 25° C. for 5 minutes, 46° C. for 20 minutes, 95° C. for 60 seconds. Bla-KPC expression was determined by qPCR, with primers designed using NCBI primer-BLAST. qPCR analysis was performed using the iTaq™ Universal SYBR Green Supermix (Bio-Rad®), in a CFX96 system by the following program: 95° C. for 3 min., 39 cycles of 95° C. for 10 sec. and 55° C. for 30 sec. Melt curves were generated for each sample by heating PCR amplicons from 65-95° C. with a gradual increase of 0.5° C./0.5 s.

### Beta-Lactamase Activity Assay

Bacterial strains were streaked from frozen LB+glycerol stocks onto LB agar plates and incubated for 24 hours at 37° C. Single colonies were grown overnight at 37° C. in 3 ml of LB. Cultures were diluted 1:1000 in 2 ml LB+128 μg/ml meropenem, and DNAzyme were added to 1.25 μM. Cultures were incubated at 37° C. with continuous shaking for 1 h. Beta-lactamase activity was measured with the beta lactamase activity assay colorimetric kit from abcam®.

### Viability Assay

HT-29 cells (ATCC) were seeded in 24-well plates at 200,000 cells per well. DNAzyme were added to the medium at different concentrations. After 72 hours, PrestoBlue™ reagent (A13262, Life Technologies Ltd.) was added as a cell viability indicator.

### Galleria mellonella Virulence Assays

Larvae virulence experiments were performed in the facilities of Pharmaseed LTD (Ness Ziona, Israel). Larvae were obtained from BioSystems Technologies (Exeter, UK). Larvae were randomly selected from a large batch for an approximate weight of 250±25 mg and a plump, light-colored morphology. Each treatment group (20 larvae per group) contained 10 larvae placed in a 10 cm empty Petri dish (2 dishes per treatment group), on filter paper.

Bacterial inoculums were prepared from cultures grown overnight at 37° C. in TSB medium (HyLabs, Israel) and washed with sterile PBS. Bacterial concentration was determined by McFarland reference kit (DENSICHEK® plus, BioMerieux) and diluted in sterile PBS to obtain 10⁹ CFU/mL. Serial dilutions of bacterial suspensions were made, and spots were plated on blood agar plates, which were incubated at 37° C. overnight, then CFU were counted to determine final injected bacterial concentrations.

Each larva was initially injected with 5×10⁵ cells into the pseudopod, followed by a second injection to the prolog of meropenem (10 μg/g), meropenem+DNAzyme (8.52 μg/g) or sterile PBS. Infected larvae and PBS-injected controls were maintained at 37° C. and monitored every 12 hours for one week. Larvae were recorded as dead if they turned grey or black and/or did not respond to touch of head with blunt forceps.

### Ex Vivo

Lungs were harvested from 5-10 mice (C57BL/6, 6-8 weeks old) and placed in petri dishes containing DMEM 5% FCS. The tissue was divided into circular samples 3 mm in diameter with a biopsy punch and transferred to poly-propanol tubes with 3 ml DMEM. DMEM contained meropenem and/or DNAzymes, as indicated. To each respective tube 10 μl of mid-logarithmic bacterial culture was added, with 3 technical repeats for each condition. Tubes were incubated at 37° C. for 24 hours and washed twice with PBS. For CFU of bacteria infecting ex vivo tissue culture, individual samples or their growth media were collected, samples were re-suspended in 1 ml PBS, and free-living bacteria were pelleted and re-suspended in PBS in the same volume. Cells colonizing the lung tissue were extracted by moderate sonication (2-4 pulses of 5 sec, amplitude 30% per samples). In all cases, to determine the number of viable cells, samples were serially diluted in PBS, plated on LB plates, and colonies were counted after incubation at 37° C. overnight.

### KP Infected Thigh Muscle Model in Neutropenic Mice

Neutropenia induction: Experiments were performed on 9-10 week-old female ICR mice. On day (−4), neutropenia was induced by intraperitoneal (IP) injection of 10 mL/kg of 150 mg/kg cyclophosphamide. At day (−1), an IP booster shot of the same volume of cyclophosphamide was given, this time at 100 mg/kg.

Bacterial suspension preparation: At day −4, KP (ATCC® BAA-1705™) bacteria were streaked out from −80° C. glycerol stock onto LB medium, incubated at 37° C. for 20 hours, and then stored at 2-8° C. At day −1, single bacterial colonies were picked by using an inoculation loop and suspended in 20 ml. Tryptic Soy broth (TSB) (Hylabs) in Erlenmeyer flasks and incubated at 37° C. overnight with shaking at 180 rpm. On the morning of the dosing day, the culture was diluted in 20 mL fresh TSB, incubated for 1-3 hours at 37° C., centrifuged at 3500 g for 10 minutes at RT, and the pellet was resuspended in sterile PBS. Bacterial concentration was determined as described hereinabove and diluted in sterile PBS to a concentration of 2.2×10⁷ CFU/mL.

Inoculum and treatment: On day 1, each mouse was injected intramuscular (IM) with 50 μL of bacterial suspension in each thigh. 15 mL/kg of 240 mg/kg meropenem was injected subcutaneously (SC). 2.5 mL/kg of 16, 24 or 32 mg/kg DNAzyme KPC-337 was injected SC every 6-8 hours, until the conclusion of the study.

Termination and Necropsy: Animals were sacrificed by CO₂ asphyxiation. Muscle tissues from femur and pelvic bones of both legs were harvested. Tissues were weighed and homogenized in 3 mL of ice-cold PBS using gentleMACS™ Dissociator.

Bacterial burden determination: Homogenate sample dilutions were plated on LB agar plates at various dilutions to quantify colony forming units (CFUs). Plates were incubated overnight (at 37° C.), and bacterial burden was calculated and expressed as CFUs/gram of tissue.

### Example 1

### Design of DNAzymes Targeting Bacterial Resistance Genes

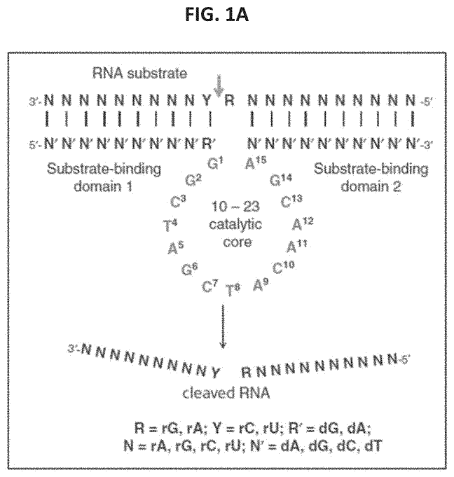

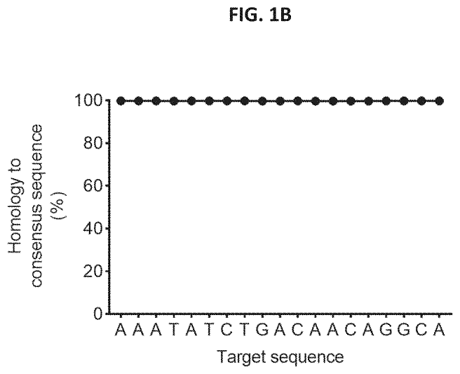

Conjugated deoxyribozymes (DNAzymes) were evaluated for ability to specifically and efficiently hydrolyze RNA transcripts of resistance genes and restore antibiotic susceptibility of resistant isolates of bacteria. This was carried out by designing DNAzymes that specifically cleave selective RNA transcripts that confer antibiotic resistance (Tables 1 and 2) and conjugating them to cholesterol via a linker. The DNAzymes consist of a catalytic core, flanked by two arms that recognize its RNA target through Watson-Crick base pairing and cleave RNA at a specific phosphodiester linkage (FIG. 1A).

### Example 2

### DNAzyme Targeting KPC Gene in Klebsiella pneumoniae

One DNAzyme (KPC-337, set forth in SEQ ID NO: 1) was designed to target a conserved region in RNA transcripts of the gene bin carbapenemase of clinically relevant strains of K. pneumoniae (ec number 3.5.2.6, uniport ID Q9F663). In total, 30% (3845/12539) of sequenced. isolates of K. pneumoniae have the annotation of bla-KPC, in which the target region (AAATATCTGACAACAGGC, bases 345-363, SEQ ID NO: 27) is conserved in 99.6% of the isolates (FIG. 1B).

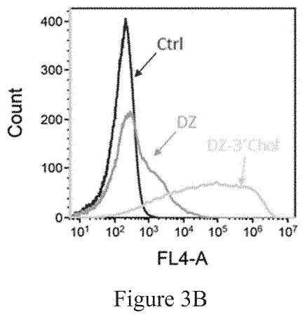

The first obstacle in applying DNAzymes as antibacterial agents is the delivery of DNAzymes into bacterial cells efficiently. DNAzyme have to overcome multiple physiological barriers from the administration site to the intracellular of the bacteria. The major hurdle for internalization of DNAzymes to the bacterial cells is the cell wall. Notably, the outer membrane of a gram-negative bacteria, which is absent in grain-positive bacteria, is the main reason for resistance to a wide range of antibiotics, due to its hydrophobic nature, which physically blocks the diffusion of some antibiotics. Conjugation of cholesterol to DNAzymes facilitated the uptake of DNAzymes into K. pneumoniae, in the presence of sub-toxic concentration of meropenem (FIGS. 3A-B).

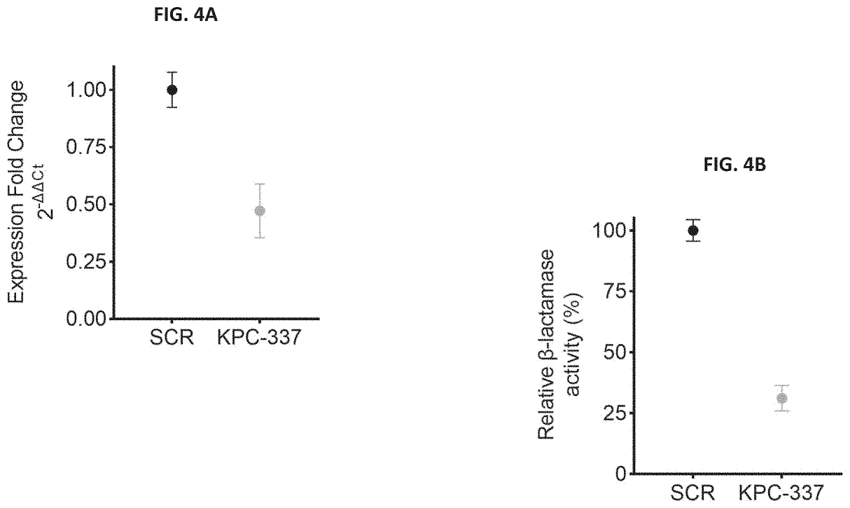

To test the bioactivity of KPC-337, the RNA and protein levels of bla-KPC were measured following combined treatment with sub-toxic concentration of meropenem. and KPC-337. Addition of the DNAzyme KPC-337 to bacterial culture of K. pneumoniae strain ATCC® BAA-1705™ reduced transcript levels of bla-KPC by two-fold (FIG. 4A) and beta-lactamase activity by 70% (FIG. 4B). All experiments included a control that consisted of the same nucleotides as KPC-337, 3′-conjugated to cholesterol-TEG, but in a random non catalytic order, designated as “scramble” (SCR). The SCR control did not alter RNA levels nor beta-lactamase activity from untreated control levels. This indicates that the DNAzyme is capable of entering the bacteria and binding to and specifically cleaving the intracellular RNA transcripts.

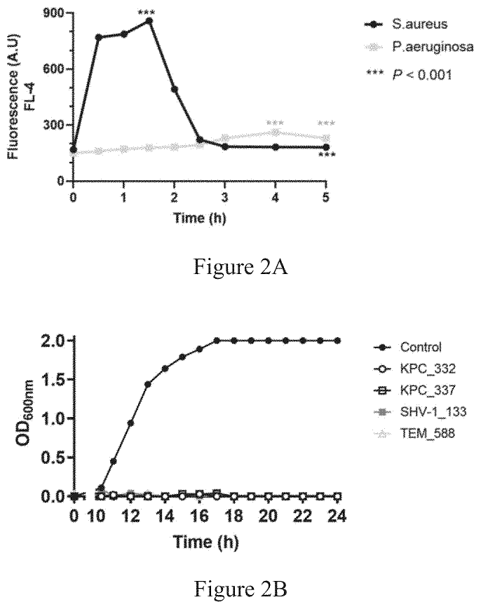

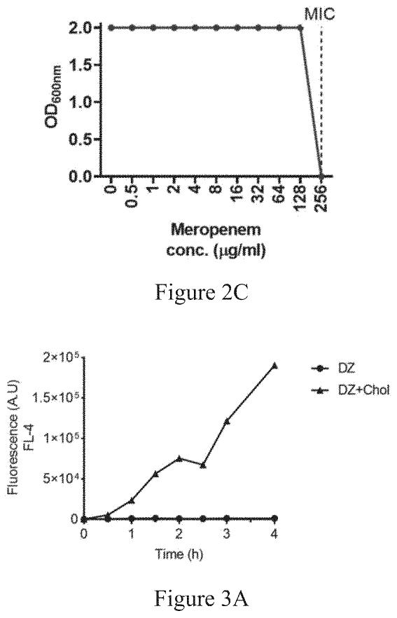

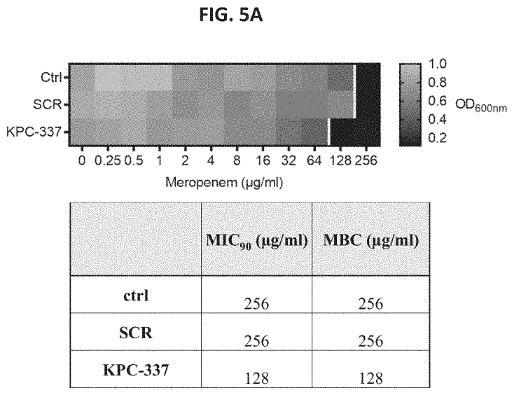

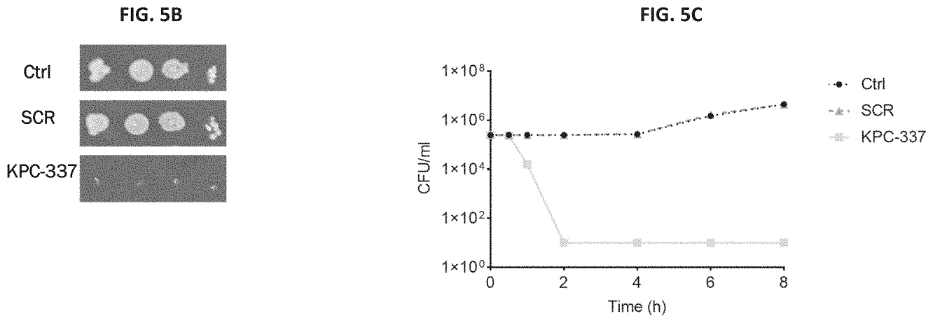

KPC-337 targets a conserved region in bla carbapenemase RNA transcripts that confers carbapenem antibiotic resistance in carbapenem-resistant K. pneumoniae (CRKP). Addition of KPC-337 to CRKP culture reduced the MIC levels by two fold (from 256 μg/ml to 128 μg/ml; FIGS. 2C and 5A). The combined treatment of meropenem and KPC-337 was bactericidal, (FIGS. 5B-C).

Additional DNAzymes that target resistance genes (e.g., KPC, TEM, SHV-1 and OXA-18, set forth in SEQ ID -Nos: 2-4 and 31-49) were tested for meropenem sensitization of ATCC® BAA-1705™, and four of the tested DNAzymes, KPC-332, KPC-337, TEM-588, and SHV1-133, inhibited bacterial growth (FIG. 2B).

### Example 3

### Ex Vivo Effect of a DNAzyme Targeting KPC Gene in Klebsiella pneumoniae

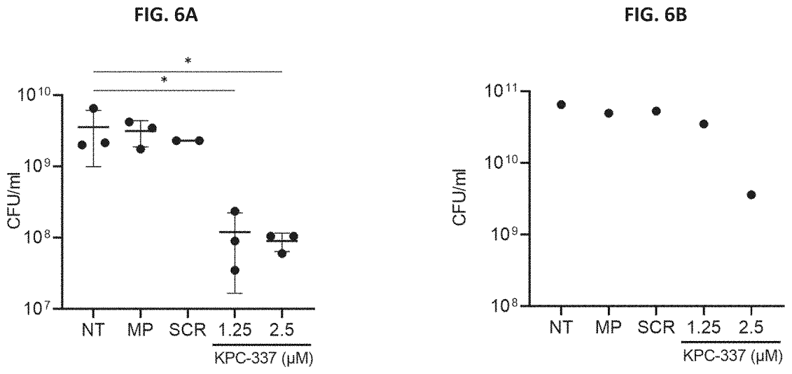

K. pneumoniae is a vital pathogen of community and hospital-acquired pneumonia. A lung ex-vivo infection model was used to test the effect of the DNAzymes during the initial infection stages. Treatment with sub-toxic levels of meropenem with KPC-337 reduced the capacity of the bacteria to infect and colonize lung tissue by approximately 1.5-2 fold (FIG. 6A). In addition, a further 1-1.5-fold reduction of the free-living cells that remained in the growth media was also observed (FIG. 6B). Treatment with meropenem alone or in combination with the scramble control (SCR) did not reduce the bacterial load in the tissue or in the growth media (FIG. 6B).

### Example 4

### In Vivo Effect of a DNAzyme Targeting KPC Gene in Klebsiella pneumoniae

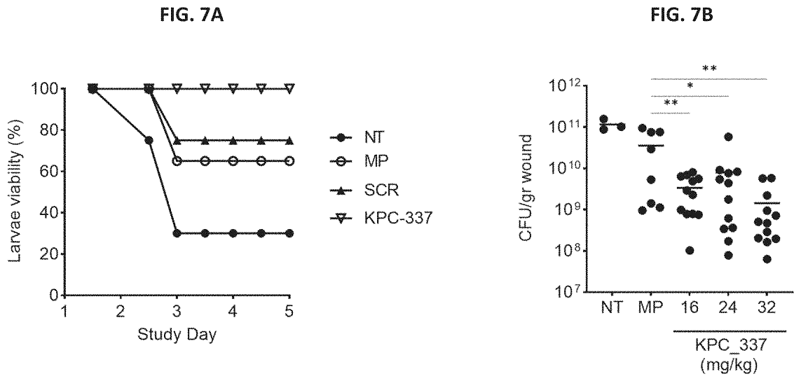

Wax moth Galleria mellonella has been utilized to study key virulence mechanisms in Klebsiella spp., such as cell death associated with bacterial replication, avoidance of phagocytosis by phagocytes, the attenuation of host defense responses, and the production of antimicrobial factors. Bacterial infection of G. mellonella larvae with K. pneumoniae strain ATCC® BAA-1705™ reduced the larval viability by 75%, while addition of a sub-toxic (10 μg/g) meropenem concentration, alone or with SCR, resulted in viability reductions of 40% and 35%, respectively. Adding 10 μg/g meropenem+KPC-337 achieved a larval viability of 100% (FIG. 7A). Thus, the combined treatment completely abrogated bacteria-mediated mortality.

Next, the effectiveness of the combined therapy was examined in the neutropenic thigh infection model. Animals were infected with K. pneumoniae in both thighs and were treated three times a day (TID) with sub-toxic levels of meropenem (240 mg/kg in this context), alone or combined with different amounts of KPC-337 (16, 24 or 32 mg/kg) via subcutaneous administration. Treatment with meropenem alone appeared to slightly reduce the bacterial load in the infection site, but the effect was not statistically significant (FIG. 7B). However, combined meropenem+KPC-337 treatment reduced bacterial loads by 2-3 log fold in the infection site (FIG. 7B). Thus, KPC-337 sensitized the bacteria to antibiotic in this in vivo model as well.

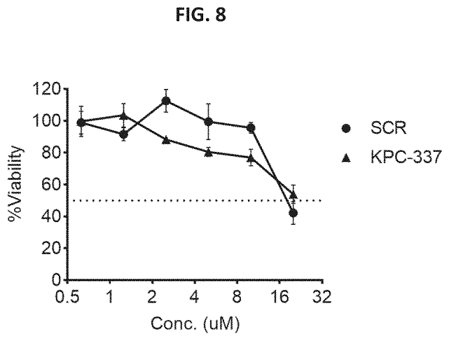

The safety of KPC-337 human cell line was further tested in HT-29 cells (a human colorectal adenocarcinoma) cell line. The IC₅₀ of toxicity of KPC-337 and SCR were both 20 μM (FIG. 8). Importantly, KPC-337 and SCR were not toxic to human cells at bioactive concentrations (≤5 μM).

### Example 5

### DNAzymes Targeting Methicillin-Resistant Staphylococcus aureus (MRSA)

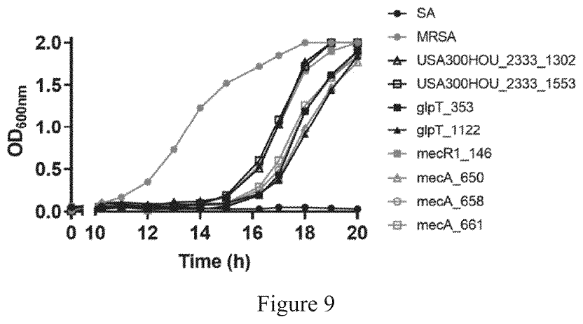

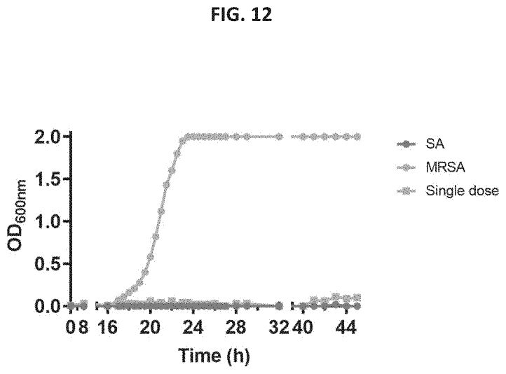

DNAzymes (set forth in SEQ ID NOs: 5-26) were designed to target a conserved region in RNA transcripts of the resistance genes mecA, mecR1, glpT, femA and USA300HOU. Conjugation of cholesterol to DNAzymes facilitated uptake of DNAzymes into the gram positive methicillin-resistant Staphylococcus aureus (MRSA) strain ATCC® BAA-1705™ and the gram negative Pseudomonas aeruginosa strain ATCC® BAA-3105™ (FIG. 2A). Addition of the DNAzymes to an ATCC® BAA-1705™ culture containing a sub-toxic concentration of cefoxitin delayed the growth of the bacteria, thus increasing the susceptibility of the bacteria to the antibiotic (FIG. 9). A time course demonstrated an inhibitory effect of growth of the bacterial culture for 44 hours, following a single dose treatment of a potent conjugated femA-targeting DNAzyme, femA-545 (set forth in SEQ ID NO: 25) in the presence of a sub-toxic concentration of cefoxitin (FIG. 12).

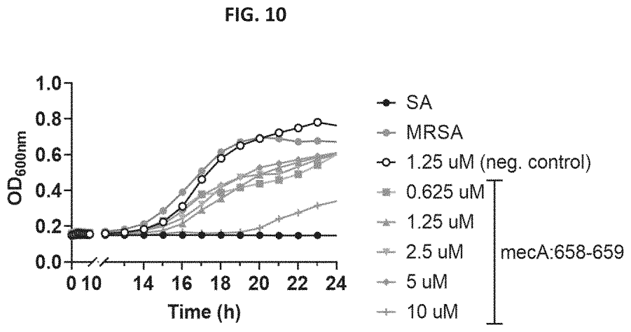

Cholesterol-conjugated DNAzyme were able to potentiate cefoxitin in a dose-dependent manner, with 10 micromolar (μM) DNAzyme exhibiting a greater effect than lower concentrations (FIG. 10).

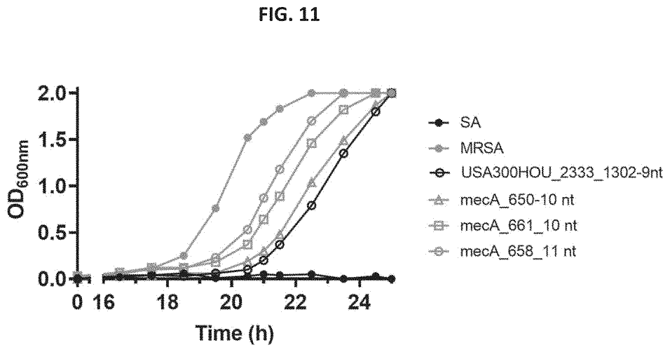

Further studies showed that conjugated DNAzymes of various arm lengths were effective at inhibiting bacterial growth and potential antibiotics (FIG. 11).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method of treating a bacterial infection in a subject in need thereof, comprising administering to said subject a DNAzyme conjugated to an organic moiety, wherein said organic moiety is selected from:
a) a cyclic organic compound having multiple rings; and
b) an alkyl compound.

2. The method of claim 1, wherein said organic moiety is a cyclic organic compound having multiple rings, and said cyclic organic compound is a steroid.

3. The method of claim 2, wherein said steroid is a sterol.

4. The method of claim 2, wherein said steroid is a bile acid or bile alcohol.

5. The method of claim 3, wherein said sterol is selected from the group consisting of cholesterol, β-sitosterol, campesterol, stigmasterol, and ergosterol; and derivatives of any of aforementioned compounds with 2 or fewer substitutions.

6. The method of claim 1, wherein said organic moiety is an alkyl compound, and said alkyl compound is a fatty acid, comprising a chain of 8-20 carbons.

7. The method of claim 1, wherein said DNAzyme is conjugated to said organic moiety via a linker, and wherein said linker is an organic moiety.

8. The method of claim 1, wherein said DNAzyme is targeted to a transcript of a bacterial gene.

9. The method of claim 8, wherein said bacterial gene is an antibiotic resistance gene.

10. The method of claim 1, wherein said DNAzyme is a 10-23 type DNAzyme molecule.

11. The method of claim 1, wherein said bacterial infection is an infection with a Gram positive bacteria.

12. The method of claim 1, wherein said bacterial infection is an infection with a Gram negative bacteria.

13. The method of claim 1, wherein said bacterial infection is an infection with selected from the group consisting of Enterococcus faecium, Staphylococcus aureus, Klebsiella pneumoniae, Acinetobacter baumannii, Pseudomonas aeruginosa, and an Enterobacter.

14. The method of claim 1, wherein said subject is a human subject.

15. A method of increasing susceptibility of a bacterium to an antibiotic, in a subject having a bacterial infection, said method comprising administering to said subject a DNAzyme conjugated to an organic moiety, wherein said organic moiety is selected from:
a) a cyclic organic compound having multiple rings; and
b) an alkyl compound.

16. The method of claim 15, wherein said organic moiety is a cyclic organic compound having multiple rings, and said cyclic organic compound is a steroid.

17. The method of claim 16, wherein said steroid is a sterol.

18. The method of claim 15, wherein said DNAzyme is conjugated to said organic moiety via a linker, and wherein said linker is an organic moiety.

19. The method of claim 15, wherein said DNAzyme is targeted to a transcript of a bacterial gene.

20. The method of claim 19, wherein said bacterial gene is an antibiotic resistance gene.


SEQUENCE LISTING (49 sequences)

SEQ ID NO: 1 - DNA, 32 bp, synthetic construct
  source 1..32: mol_type = other DNA; note = KPC_337
gcctgttgggctagctacaacgacagatattt

SEQ ID NO: 2 - DNA, 33 bp, synthetic construct
  source 1..33: mol_type = other DNA; note = KPC_332
gttgtcagaggctagctacaacgaatttttccg

SEQ ID NO: 3 - DNA, 33 bp, synthetic construct
  source 1..33: mol_type = other DNA; note = SHV-1_133
ccagatccaggctagctacaacgattctatcat

SEQ ID NO: 4 - DNA, 32 bp, synthetic construct
  source 1..32: mol_type = other DNA; note = TEM_588
tagagtaagggctagctacaacgaagttcgcc

SEQ ID NO: 5 - DNA, 33 bp, synthetic construct
  source 1..33: mol_type = other DNA; note = USA300HOU_2333_1302_9nt
ttttagttgggctagctacaacgagttagtact

SEQ ID NO: 6 - DNA, 35 bp, synthetic construct
  source 1..35: mol_type = other DNA; note = mecA_650_10nt
tattttagcaggctagctacaacgaagtcatttaa

SEQ ID NO: 7 - DNA, 35 bp, synthetic construct
  source 1..35: mol_type = other DNA; note = mecA_661_10nt
tgactcataaggctagctacaacgatattttagca

SEQ ID NO: 8 - DNA, 37 bp, synthetic construct
  source 1..37: mol_type = other DNA; note = mecA-658_11nt
tgactcataatggctagctacaacgatttagcatagt

SEQ ID NO: 9 - DNA, 33 bp, synthetic construct
  source 1..33: mol_type = other DNA; note = USA300HOU_2396: 437_9nt
gcttttttaggctagctacaacgatgactaatg

SEQ ID NO: 10 - DNA, 35 bp, synthetic construct
  source 1..35: mol_type = other DNA; note = USA300HOU_2396: 437_10nt
agcttttttaggctagctacaacgatgactaatgg

SEQ ID NO: 11 - DNA, 33 bp, synthetic construct
  source 1..33: mol_type = other DNA; note = mecA_647_9nt
ttagcatagggctagctacaacgacatttaaat

SEQ ID NO: 12 - DNA, 37 bp, synthetic construct
  source 1..37: mol_type = other DNA; note = mecA_647-11nt
ttttagcatagggctagctacaacgacatttaaataa

SEQ ID NO: 13 - DNA, 33 bp, synthetic construct
  source 1..33: mol_type = other DNA; note = mecA_650_9nt
attttagcaggctagctacaacgaagtcattta

SEQ ID NO: 14 - DNA, 37 bp, synthetic construct
  source 1..37: mol_type = other DNA; note = mecA_650_11nt
ttattttagcaggctagctacaacgaagtcatttaaa

SEQ ID NO: 15 - DNA, 33 bp, synthetic construct
  source 1..33: mol_type = other DNA; note = mecA_661_9nt
gactcataaggctagctacaacgatattttagc

SEQ ID NO: 16 - DNA, 33 bp, synthetic construct
  source 1..33: mol_type = other DNA; note = glpT_1122_9nt
tttagtaagggctagctacaacgatctttatgg

SEQ ID NO: 17 - DNA, 35 bp, synthetic construct
  source 1..35: mol_type = other DNA; note = glpT_1122_10nt
atttagtaagggctagctacaacgattatgggtac

SEQ ID NO: 18 - DNA, 33 bp, synthetic construct; identical to SEQ ID NO: 5
  source 1..33: mol_type = other DNA; note = USA300HOU_2333_1302_9nt

SEQ ID NO: 19 - DNA, 35 bp, synthetic construct
  source 1..35: mol_type = other DNA; note = USA300HOU_2333_1302_10nt
tttttagttgggctagctacaacgagttagtactc

SEQ ID NO: 20 - DNA, 33 bp, synthetic construct
  source 1..33: mol_type = other DNA; note = mecA-658_9nt
tcataattaggctagctacaacgatttagcata

SEQ ID NO: 21 - DNA, 33 bp, synthetic construct
  source 1..33: mol_type = other DNA; note = mecR1_146
gaagtcgtgggctagctacaacgacagatacat

SEQ ID NO: 22 - DNA, 31 bp, synthetic construct
  source 1..31: mol_type = other DNA; note = mecA_353
gttcgttgtccgagccggacgacgaataatt

SEQ ID NO: 23 - DNA, 33 bp, synthetic construct; identical to SEQ ID NO: 9
  source 1..33: mol_type = other DNA; note = USA300HOU_2396: 437

SEQ ID NO: 24 - DNA, 33 bp, synthetic construct
  source 1..33: mol_type = other DNA; note = USA300HOU_2333:676
atttatttaggctagctacaacgaaaaatttac

SEQ ID NO: 25 - DNA, 33 bp, synthetic construct
  source 1..33: mol_type = other DNA; note = femA_545
tttttcgtgggctagctacaacgattctttttc

SEQ ID NO: 26 - DNA, 33 bp, synthetic construct; identical to SEQ ID NO: 20
  source 1..33: mol_type = other DNA; note = mecA_658_9nt

SEQ ID NO: 27 - DNA, 18 bp, Klebsiella pneumoniae
  source 1..18: mol_type = unassigned DNA; note = target region of bla gene
aaatatctgacaacaggc

SEQ ID NO: 28 - DNA, 15 bp, synthetic construct
  source 1..15: mol_type = other DNA; note = catalytic core 10-23
ggctagctacaacga

SEQ ID NO: 29 - DNA, 14 bp, synthetic construct
  source 1..14: mol_type = other DNA; note = catalytic core 8-17
tccgagccggacga

SEQ ID NO: 30 - skipped

SEQ ID NO: 31 - DNA, 33 bp, synthetic construct
  source 1..33: mol_type = other DNA; note = KPC_568
cagtttttgggctagctacaacgaaagctttcc

SEQ ID NO: 32 - DNA, 33 bp, synthetic construct
  source 1..33: mol_type = other DNA; note = KPC_36
catgagagaggctagctacaacgaaagacagca

SEQ ID NO: 33 - DNA, 31 bp, synthetic construct
  source 1..31: mol_type = other DNA; note = KPC_470
gaacgtggggctagctacaacgaatcgccga

SEQ ID NO: 34 - DNA, 32 bp, synthetic construct
  source 1..32: mol_type = other DNA; note = KPC_389
cggcgttaggctagctacaacgacactgtatt

SEQ ID NO: 35 - DNA, 33 bp, synthetic construct
  source 1..33: mol_type = other DNA; note = KPC_563
ttttgtaagggctagctacaacgatttccgtca

SEQ ID NO: 36 - DNA, 33 bp, synthetic construct; identical to SEQ ID NO: 2
  source 1..33: mol_type = other DNA; note = KPC_332

SEQ ID NO: 37 - DNA, 33 bp, synthetic construct
  source 1..33: mol_type = other DNA; note = KPC_574
cagtgtcagggctagctacaacgattttgtaag

SEQ ID NO: 38 - DNA, 31 bp, synthetic construct
  source 1..31: mol_type = other DNA; note = KPC_633
gtgtttcctccgagccggacgattagccaat

SEQ ID NO: 39 - DNA, 32 bp, synthetic construct
  source 1..32: mol_type = other DNA; note = KPC_344
ccgtcatgggctagctacaacgactgttgtca

SEQ ID NO: 40 - DNA, 33 bp, synthetic construct
  source 1..33: mol_type = other DNA; note = OXA-18_294
atagacttgggctagctacaacgattgtatgtg

SEQ ID NO: 41 - DNA, 32 bp, synthetic construct
  source 1..32: mol_type = other DNA; note = OXA-18_59
ccacggaaggctagctacaacgatgattggga

SEQ ID NO: 42 - DNA, 33 bp, synthetic construct
  source 1..33: mol_type = other DNA; note = OXA-18_125
tataaggtaggctagctacaacgattccggtaa

SEQ ID NO: 43 - DNA, 32 bp, synthetic construct
  source 1..32: mol_type = other DNA; note = OXA-18_225
tttggcgaggctagctacaacgatgcaagatt

SEQ ID NO: 44 - DNA, 33 bp, synthetic construct
  source 1..33: mol_type = other DNA; note = SHV-1_127
ccatttctaggctagctacaacgacatgcctac

SEQ ID NO: 45 - DNA, 33 bp, synthetic construct
  source 1..33: mol_type = other DNA; note = SHV-1_33
ggtggctaaggctagctacaacgaagggagata

SEQ ID NO: 46 - DNA, 33 bp, synthetic construct
  source 1..33: mol_type = other DNA; note = SHV-1_197
ttaaaggtgggctagctacaacgatcatcatgg

SEQ ID NO: 47 - DNA, 32 bp, synthetic construct
  source 1..32: mol_type = other DNA; note = TEM_518
gctcgtcgggctagctacaacgattggtatgg

SEQ ID NO: 48 - DNA, 33 bp, synthetic construct
  source 1..33: mol_type = other DNA; note = TEM_810
tctatttcgggctagctacaacgatcatccata

SEQ ID NO: 49 - DNA, 33 bp, synthetic construct
  source 1..33: mol_type = other DNA; note = TEM_14
acacggaaaggctagctacaacgagttgaatac